SEQUENCE LISTING (11 sequences)

SEQ ID NO: 1 - protein, 1251 aa, Clostridium butyricum
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNCTMNFKNNNGNNIGLLGFKADTVVASTWYYTHMRDNTNSNGFFWNFISEEHGWQEK

SEQ ID NO: 2 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 4 - protein, 1297 aa, Clostridium botulinum
  misc_feature (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 5 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 6 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 7 - protein, 1274 aa, Clostridium botulinum
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 8 - protein, 1291 aa, Clostridium botulinum; identical to SEQ ID NO: 2

SEQ ID NO: 9 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 10 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 11 - protein, 1296 aa, Clostridium botulinum [sequence omitted]

(19) United States
(12) Patent Application Publication
(10) Pub. No.: US 2012/0189677 A1
(43) Pub. Date: Jul. 26, 2012

(54) FORMULATIONS

(76) Inventors: Stephen TONGE, (US); Andrew HARPER, (US)

(21) Appl. No.: 13/354,440

(22) Filed: Jan. 20, 2012

Related U.S. Application Data
(60) Provisional application No. 61/434,579, filed on Jan. 20, 2011.

(51) Int. Cl. A61K 8/02; A61K 38/48; A61K 9/127; A61K 8/66; A61K 9/00; A61K 8/14; B82Y 40/00; B82Y 5/00

(52) U.S. Cl. 424/401; 424/400; 424/94.67; 424/450; 977/797; 977/906; 977/907; 977/840

(57) ABSTRACT

A formulation comprising botulinum toxin (BT), lipid and surfactant, characterised in that the lipid and surfactant are in the form of macromolecular assemblies of less than 100 nm in diameter. The surfactant may have an HLB number of less than 20. Cosmetic and pharmaceutical formulations and corresponding uses are contemplated and included, as are methods of preparation,

The present invention relates inter alia to formulations of botulinum toxin which are of use in the cosmetic or pharmaceutical fields. Also provided are low temperature methods for the manufacture of formulations, such as those containing botulinum toxin.

Clostridium botulinum, an anaerobic, gram-positive bacterium, produces a potent polypeptide neurotoxin known as botulinum toxin (BT). BT causes a neuroparalytic illness in humans and animals referred to as botulism.

BT acts by preventing synaptic transmission at neuromuscular junctions. Blocking of the signals that normally would cause muscle spasms or contractions results in a flaccid paralysis. In particular, BT blocks the exocytosis of acetylcholine by cleaving proteins that are essential for the fusion of synaptic vesicles with the presynaptic membrane.

BT may be purified from Clostridium botulinum culture or recombinantly produced. BT is available commercially from a number of suppliers, such as Metabiologics, Inc. (Wisconson, USA) or List Biological Laboratories, Inc. (California, USA).

### SUMMARY OF THE INVENTION

The present invention provides a formulation comprising BT, lipid and surfactant, characterised in that the lipid and surfactant are in the form of macromolecular assemblies of less than 100 nm in diameter.

According to the present invention there is provided a formulation comprising BT, lipid and surfactant, characterised in that the surfactant has an HLB number of less than 20 and in that the lipid and surfactant are in the form of macromolecular assemblies of less than 100 nm in diameter.

Also there is provided a formulation comprising BT, lipid and surfactant, characterised in that the surfactant has an HLB number in the range of about 10.5 to about 17.5 and in that the lipid and surfactant are in the form of macromolecular assemblies of less than 100 nm in diameter.

Further, there is provided a formulation comprising BT, lipid and surfactant, characterised in that the surfactant is an ether surfactant and in that the lipid and surfactant are in the form of macromolecular assemblies of less than 100 nm in diameter.

Additionally, there is provided a formulation comprising BT, lipid and surfactant, characterised in that the surfactant is an ester surfactant and in that the lipid and surfactant are in the form of macromolecular assemblies of less than 100 nm in diameter.

There is also provided, a formulation comprising BT, lipid and surfactant, characterised in that the surfactant is an ionic surfactant and in that the lipid and surfactant are in the form of macromolecular assemblies of less than 100 nm in diameter.

Also provided are formulations comprising BT, lipid and a copolymer of styrene and maleic acid wherein the polymer and lipid are in the form of macromolecular assemblies of less than 100 nm in diameter.

Such compositions may be referred to herein as formulations of the invention.

There is provided a cosmetic preparation comprising a formulation of the invention and a cosmetically acceptable carrier or excipient.

There is also provided a pharmaceutical preparation comprising a formulation of the invention and a pharmaceutically acceptable carrier or excipient.

### BRIEF DESCRIPTION OF THE FIGURES

FIG. 1 provides an illustration of the turbidity of samples prepared in Example 1 of WO2008/065451 using ethoxyalkylated aromatic alcohol ether surfactants.

FIG. 2 provides an illustration of the turbidity of samples prepared in Example 1 herein, using a low temperature process and ethoxyalkylated aromatic alcohol ether surfactants.

FIG. 3 is the particle size analysis for an aqueous composition containing the surfactant Brij35P (7.0% w/w), the lipid S-75 (1% w/w) and maltodextrin (2% w/w), prepared using a low temperature process—principal particle size 8.68 nm, polydispersity 0.625.

FIG. 4 provides the results of a skin penetration study involving macromolecular assemblies of the type used in the present invention.

FIG. 5 provides an alignment of a number of exemplary natural BT sequences obtained from the Uniprot database.

FIGS. 6a-c Show the results of testing formulations of the invention in a hydrosis model.

### DESCRIPTION OF SEQUENCES

SEQ ID No: 1 Polypeptide sequence for a BT type A obtained from the Uniprot database (Accession number P10845).

SEQ ID No: 2 Polypeptide sequence for a BT type B obtained from the Uniprot database (Accession number B1INP5).

SEQ ID No: 3 Polypeptide sequence for a BT type A obtained from the Uniprot database (Accession number Q45894).

SEQ ID No: 4 Polypeptide sequence for a BT type G obtained from the Uniprot database (Accession number Q60393).

SEQ ID No: 5 Polypeptide sequence for a BT type Cl obtained from the Uniprot database (Accession number P18640).

SEQ ID No: 6 Polypeptide sequence for a BT type A obtained from the Uniprot database (Accession number A5 HZZ9).

SEQ ID No: 7 Polypeptide sequence for a BT type F obtained from the Uniprot database (Accession number P30996).

SEQ ID No: 8 Polypeptide sequence for a BT type B obtained from the Uniprot database (Accession number P10844).

SEQ ID No: 9 Polypeptide sequence for a BT type D obtained from the Uniprot database (Accession number P19321).

SEQ ID No: 10 Polypeptide sequence for a BT type E obtained from the Uniprot database (Accession number Q00496).

SEQ ID No: 11 Polypeptide sequence for a BT type A obtained from the Uniprot database (Accession number P10845).

### DETAILED DESCRIPTION OF THE INVENTION

The present invention relates to formulations comprising BT, a lipid and a surfactant wherein the lipid and surfactant are in the form of macromolecular assemblies. Other aspects relate to formulations comprising BT, lipid and a copolymer of styrene and maleic acid wherein the polymer and lipid are in the form of macromolecular assemblies.

### Botulinum Toxin

Clostridium botulinum, an anaerobic, gram-positive bacterium, produces a potent polypeptide neurotoxin known as botulinum toxin (BT). BT causes a neuroparalytic illness in humans and animals referred to as botulism.

A number of serologically distinct types of BT are known to exist, classified by the designations A, B, C, D, E, F and G. Most of these designations encompass a plurality of subtypes (e.g. at least five A subtypes are known). A high degree of sequence homology exists between toxin types. All BT are initially produced as relatively inactive, single polypeptide chains with a molecular mass of about 150 kDa which is susceptible to proteolytic cleavage to yield an activated form. The activated form consists of a heavy chain (HC) of roughly 100 kDa and a light chain (LC) of roughly 50 kDa which are linked by a disulfide bond.

The toxin is normally found within a complex where it is associated with various other nontoxic proteins, which may also have hemagglutinating properties. The complex is believed to stabilise the BT and protect against proteolysis.

BT acts by preventing synaptic transmission at neuromuscular junctions. Blocking of the signals that normally would cause muscle spasms or contractions results in a flaccid paralysis. In particular, BT blocks the exocytosis of acetylcholine by cleaving proteins that are essential for the fusion of synaptic vesicles with the presynaptic membrane.

The C-terminal portion of the heavy chain (HC) binds selectively and irreversibly to high affinity receptors at the presynaptic surface of cholinergic neurones—vesicle proteins which have been exposed at the cell surface by exocytoticfusion of synaptic vesicles. The particular receptor protein which is targeted is dependent upon the BT type—a luminal domain of synaptic vesicle protein 2 (A, B and C) is the target for BT type A, whereas an intravesicular region of synaptotagmin (I and II) acts as a receptor for BT type B and type G.

As synaptic vesicle proteins are recovered from the plasma membrane, the BT is carried into the lumen of recycling vesicles. Acidification of the new vesicle induces translocation of the light chain (LC) into the presynaptic cytosol via a membrane-spanning channel formed by the HC. The LC is subsequently released by reduction of the disulfide bond in the cytoplasm.

The LC chain is a zinc protease which selectively cleaves proteins essential for recognition and docking of neurotransmitter-containing vesicles with the cytoplasmic surface of the pre-synaptic membrane, and subsequent fusion of the vesicles with the plasma membrane. BT type A and type E cleave SNAP-25 (synaptosome-associated protein of 25 kDa), but at different locations within the protein. BT type B, type D, type F and type G cause degradation of vesicle-associated membrane protein, also known as synaptobrevin, again with each BT type cleaving the protein at a different site. BT type C1 cleaves both syntaxin and SNAP-25.

The effect of BT is temporary and nerve-muscle communication is restored over the course of several months.

BT may be purified from Clostridium botulinum culture or recombinantly produced. BT is available commercially from a number of suppliers, such as Metabiologics, Inc. (Wisconson, USA) or List Biological Laboratories, Inc. (California, USA).

BT is an important agent in both the cosmetic and pharmaceutical fields. Many hyperexcitability disorders of cholinergically innervated muscles are treatable with BT including strabismus, blepharospasm and focal dystonias, hemifacial spasm and various spastic movement disorders. Clinical reports have also been published for other uses, pain relief, such as headaches, hypersalivation and hyperhydrosis.

Cosmetic use of BT includes: the correction of lines, creases and wrinkling over the entire facial area, chin, neck and chest; treatment of the depressor anguli oris, nasolabial folds, mentalis, medial and lateral brow lifts, to lessen shadows and maintain a smooth appearance.

Marketed products include Botox® (Allergan, Calif., USA), Dysport® (Ipsen, UK), Xeomin® (Merz Pharmaceuticals GmbH, Germany) which are all derived from BT type A. The proportion of complexed proteins varies among the three presentations, as does the relative effectiveness of 1 unit of the toxin (one unit of BT being defined as the LD₅₀ upon intraperitoneal injection into female Swiss Webster mice weighing 18 to 20 grams each). Neurobloc®/Myobloc® (Solstice Neurosciences LLP, California, USA) is a BT type B preparation. All of these products are intended for administration by injection directly into the area to be treated.

Topical BT formulations developed by Revance Therapeutics, Inc. (California, USA) are undergoing clinical trials, see for example WO2006/094263 and WO2007/059528. Topical administration of botulinum toxin may advantageously prevent neurotoxin passing into the circulatory system of the patient, reduce or eliminate pain associated with injections and reduce the likelihood of infection.

A number of parties have made use of the essentially ‘modular’ nature of BT to create synthetic constructs which have properties derived from various parent sequences. For example, chimeras of BT type A and BT type E have been created by swapping the C-terminal portions of the HC, resulting in an apparent transfer of some of the biochemical differences seen with the different natural BT types (Wang J et al. Journal of Biological Chemistry 2008 283(25):16993-17002).

Organisations such as the Health Protection Agency and Syntaxin Limited (Abingdon, UK) have gone further and, mimicking the manner in which BT works, have created synthetic constructs with three key domains (i) a recognition/targeting domain (analogous to the BT HC C-terminal) (ii) translocation domain (analogous to the BT HC N-terminal) and a proteolytic domain (analogous to the BT LC), see for example WO94/21300, WO96/33273, WO98/07864, WO01/58936, WO2005/23309, WO2006/59093, WO2006/59105, WO2006/59113, WO2007/138336, WO2007/138339, WO2009/150469, WO2009/150470 and WO2010/020811. The approach enables particular disorders to be addressed by modifying the targeting domain to focus on associated cell types.

### Current Use of BT for Hyperhydrosis Treatment

Eccrine sweat glands are under cholinergic nervous control and stimulation results in sweating (hydrosis). Excessive sweating results from over activity of the eccrine system or lack of feedback control and leads to an unpleasant condition for the sufferer known as hyperhydrosis which is currently treated with intradermal injection of botulinum toxin subtype A (BT-A) directly into the affected areas e.g. palms of hand, soles of the feet or underarm area (axillae). This treatment can be both painful requiring regional or topical anaesthesia, inconvenient and a temporary solution to the problem. Hence a topical alternative would offer a distinct advantage for the patient, being pain free, rapid and convenient to apply and not require specialist application.

BT-A exerts its effect on the eccrine sweat glands by inhibiting the cholinergic innervation by preventing the exocytosis of acetylcholine and thereby reduces sweat production.

In order to assess the efficacy of BT-A to inhibit sweating (hydrosis) the affected area of skin is first visualized with iodine starch staining (Minor test). Then 50-200 units of BT-A are injected intradermally; the dose is divided into 10-15 aliquots injected at spatial intervals of approximately 2 cm, enough to cover the entire treatment area.

### The HLB System

In order to function as a surfactant, a compound must necessarily include at least one hydrophilic moiety (polar or charged) and at least one hydrophobic/lipophilic moiety (non-polar). The HLB system provides an empirical parameter often assigned to a surfactant in order to characterise its hydrophilic/hydrophobic balance (see Griffin, W C Journal of the Society of Cosmetic Chemists 1949: 1:311-326; Griffin W C Journal of the Society of Cosmetic Chemists 1954 5:249-256; Florence A T et al Physiochemical Principles of Pharmacy, Chapman & Hall, London, England, 1982 (in particular pages 234-235); Aulton M E Pharmaceutics—The Science of Dosage Form Design, Churchill Livingstone, 2002 (in particular Chapter 6 pages 96-97, Chapter 23 pages 345-347)). Surfactants having higher HLB values are generally more hydrophilic, with those having lower HLB values generally being more hydrophobic.

The HLB of polyhydric alcohol fatty acid esters such as glycerol monostearate may be obtained from the equation:

HLB=20[1−(S/A)]

where S is the saponification number of the ester and A is the acid number of the fatty acid. Based on this relationship, the HLB of polyoxyethylene-20 sorbitan monolaurate is determined to be 16.7 (S being 45.5, A being 276).

In the case of materials for which it is not possible to determine saponification numbers, HLB is calculated from:

HLB=(E+P)/5

where E is the percentage by weight of oxyethylene chains and P is the percentage by weight of polyhydric alcohol groups (glycerol or sorbitol). If the hydrophile consists only of oxyethylene groups, the HLB equation may be simplified to:

HLB=(E)/5

Calculation of the contributions made by the various functional groups present within the molecule is possible using the formula:

HLB=[(sum of hydrophilic group numbers)−(sum of lipophilic group numbers)]+7

where the group numbers associated with specific moieties have been determined quantitatively (see Davies J T et al Interfacial Phenomena, Academic Press, New York, 1961).

Although the HLB system was developed for application to non-ionic surfactants, it is possible to estimate equivalent numbers for ionic surfactants by taking account of the hydrophilic contribution of the ionic groups under given conditions. Sodium lauryl sulphate (also known as SDS) is considered to be among the most potent of common detergents (McCutcheon's Volume 1: Emulsifiers & Detergents, International Edition, MC Publishing Company, Glen Rock, N.J., USA, 2005).

The HLB of a mixture of two surfactants containing fraction f of component A and (1-f) of component B is an algebraic mean of the two HLB numbers:

HLBₘᵢₓₜᵤᵣₑ=f[HLB_{A}]+(1−f)[HLB_{A}]

Additionally, it should be noted that many commercial surfactant products are not pure compounds, rather being complex mixtures of compounds, and the HLB value reported in the literature for a particular surfactant may more accurately be characteristic of a commercial product of which the compound is the major component. As a result, commercial products having the same primary surfactant component can have slightly different HLB values when sourced from different suppliers, due to manufacturing variations which lead to the presence of different impurities and quantities thereof. Variation can also occur, to some degree, between different batches obtained from the same supplier (particularly where the surfactants are derived from a mixture of natural products, for example, castor oil or lanolin based surfactants).

HLB theory is explained quantitatively by lsraelachvili J N Intermolecular and Surface Forces, 2^{nd} edition, Academic Press, London, 1991, using a theory of critical packing parameters defined by:

P=v/(a₀l_{c})

where P is the critical packing parameter (defining the ‘shape’ of the surfactant assembly—cone, truncated cone, cylinder or inverted truncated cone), v is the volume of the hydrophobic chain, a_{z}, is the surface area of the polar headgroup and l_{c} is the critical chain length of the hydrophobic tail of the surfactant.

The HLB values for a range of surfactants are provided in the Examples of WO2008/065451.

International patent applications WO99/009955 and WO2006/129127, disclose compositions comprising a lipid and copolymer of styrene and maleic acid wherein the polymer and lipid are in the form of macromolecular assemblies. International patent application WO2008/065451 discloses compositions comprising a lipid and a surfactant, wherein the surfactant and lipid are in the form of macromolecular assemblies.

As a protein which is usually found in solution, the skilled person would not reasonably expect that BT could be used in conjunction with macromolecular assemblies of the type described in WO99/009955, WO2006/129127 and WO2008/065451 (each of which is specifically incorporated herein by reference). Nevertheless, the present inventors have combined BT with such macromolecular assemblies to surprising effect.

BT formulations of the present invention may have one or more of the following advantages compared to the approaches of the prior art:
(i) be more stable (in dried and/or in aqueous form);
(ii) result in less irritation/undesirable side effects;
(iii) facilitate penetration through the skin without the use of needles;
(iv) provide a rapid onset of action;
(v) have clinical efficacy at lower dosage levels;
(vi) be easily and economically produced (e.g. utilising few components and/or inexpensive components);
(vii) contain only cosmetically/pharmaceutically acceptable components;
(viii) contain only components of natural and/or non-animal origin.

The present invention provides a formulation comprising BT, lipid and surfactant, characterised in that the lipid and surfactant are in the form of macromolecular assemblies of less than 100 nm in diameter.

According to the present invention there is provided a formulation comprising BT, lipid and surfactant, characterised in that the surfactant has an HLB number of less than 20 and in that the lipid and surfactant are in the form of macromolecular assemblies of less than 100 nm in diameter.

Also there is provided a formulation comprising BT, lipid and surfactant, characterised in that the surfactant has an HLB number in the range of about 10.5 to about 17.5 and in that the lipid and surfactant are in the form of macromolecular assemblies of less than 100 nm in diameter.

Further, there is provided a formulation comprising BT, lipid and surfactant, characterised in that the surfactant is an ether surfactant and in that the lipid and surfactant are in the form of macromolecular assemblies of less than 100 nm in diameter.

Additionally, there is provided a formulation comprising BT, lipid and surfactant, characterised in that the surfactant is an ester surfactant and in that the lipid and surfactant are in the form of macromolecular assemblies of less than 100 nm in diameter.

There is also provided, a formulation comprising BT, lipid and surfactant, characterised in that the surfactant is an ionic surfactant and in that the lipid and surfactant are in the form of macromolecular assemblies of less than 100 nm in diameter.

Also provided are formulations comprising BT, lipid and a copolymer of styrene and maleic acid wherein the polymer and lipid are in the form of macromolecular assemblies of less than 100 nm in diameter.

Such compositions may be referred to herein as formulations of the invention.

There is provided a cosmetic preparation comprising a formulation of the invention and a cosmetically acceptable carrier or excipient.

There is also provided a pharmaceutical preparation comprising a formulation of the invention and a pharmaceutically acceptable carrier or excipient.

Also provided are methods for the manufacture of a formulation of the invention comprising the steps of:
(i) Preparing an aqueous emulsion of lipid and BT; and
(ii) Mixing surfactant (optionally in aqueous solution) with the aqueous lipid/BT emulsion;
such that macromolecular assemblies are formed.

Also provided are methods for the manufacture of a formulation of the invention comprising the step mixing the macromolecular assemblies of lipid and surfactant, characterised in that macromolecular assemblies are less than 100 nm in diameter with BT. During mixing, the macromolecular assemblies and BT may be in dried form. Alternatively the macromolecular assemblies may be in aqueous solution and the BT may be in dried form. Additionally, the macromolecular assemblies may be in dried form and the BT may be in aqueous solution. Further, the macromolecular assemblies may be in aqueous solution and the BT may be in aqueous solution.

Additionally provided are methods for the manufacture of a composition comprising a lipid and a surfactant, wherein the lipid and surfactant are in the form of macromolecular assemblies comprising the steps of:
(i) Preparing an aqueous emulsion of maltodextrin and lipid; and
(ii) Mixing surfactant (optionally in aqueous solution) with the aqueous maltodextrin and lipid emulsion;
such that macromolecular assemblies are formed.

Further, there are provided methods for the manufacture of a formulation comprising BT, a lipid and a surfactant, wherein the lipid and surfactant are in the form of macromolecular assemblies comprising the steps of:
(i) Preparing an aqueous emulsion of maltodextrin, BT and lipid; and
(ii) Mixing surfactant (optionally in aqueous solution) with the aqueous emulsion of maltodextrin, BT and lipid;
such that macromolecular assemblies are formed.

### Surfactants

By the term surfactant when used herein is meant a surface active component which is capable of interacting with the lipid component to form the macromolecular assemblies of the invention.

The surfactant may consist of a single component, although will often be a mixture of components (typically, though not necessarily, of similar chemical structure).

Typically, the surfactant of use in the present invention will have an HLB number of less than 20, such as in the range of about 10.5 to about 17.5, suitably about 12 to about 17, more suitably about 13.5 to about 17. In one embodiment of the invention the surfactant will have an HLB which is between 12 to less than 13. In a second embodiment of the invention the surfactant will have an HLB which is between 13 to less than 14. In a third embodiment of the invention the surfactant will have an HLB which is between 14 to less than 15. In a fourth embodiment of the invention the surfactant will have an HLB which is between 15 to less than 16. In a fifth embodiment of the invention the surfactant will have an HLB which is between 16 to less than 17. In a sixth embodiment of the invention the surfactant will have an HLB which is between 17 to less than 18.

Typically the surfactant will have a molecular weight of less than about 10000 Da, suitably less than about 8000 Da, especially less than about 5000 Da, in particular less than about 3000 Da, such as less than about 2500 (e.g. less than about 1800 Da). In certain embodiments the surfactant will have a molecular weight of between 3000 to 8000 Da.

For pharmaceutical and cosmetic applications it is desirable that the surfactant selected is suitable for pharmaceutical or cosmetic use respectively (e.g. it has been approved for pharmaceutical or cosmetic use by an appropriate authority). For certain applications it is desirable that the surfactants are biodegradable (e.g. for injectable formulations). In some applications it is desirable that the surfactant is of natural origin and/or from a non-animal source (e.g. of natural origin and from a non-animal source, such as from plants).

The surfactant of use in the present invention can be ionic (such as the anionic, cationic, and amphoteric surfactant classes described below) or non-ionic (such as the ether and ester surfactant classes described below).

A number of standard texts are available which provide detailed summaries of the more common types of surfactant: McCutcheon's Volume 1: Emulsifiers & Detergents, International Edition, MC Publishing Company, Glen Rock, N.J., USA, 2005; Handbook of Industrial Surfactants, M Ash & I Ash, Gower Publishing Company, Aldershot, England, 1993; Surfactant Encyclopaedia, Cosmetics & Toiletries Resource Series, 2^{nd} Edition, M M Rieger, Allured Publishing Corporation, Carol Stream, USA, 1996.

The surfactant will typically not be silicone based.

### Ethers

In one embodiment of the invention the surfactant is an ether surfactant.

The broad class of ether surfactants may be separated into a number of sub-classes which include:
ethoxylated alcohols
propoxylated/ethoxylated ethers
polyglyceryl ethers
sugar ethers

In an embodiment of the invention of particular interest the ether surfactant is an ethoxylated alcohol. In a second embodiment of the invention the ether surfactant is a propoxylated/ethoxylated ether. In a third embodiment of the invention the ether surfactant is a polyglyceryl ether. In a fourth embodiment of the invention the ether surfactant is a sugar ether.

Ethoxylated alcohol surfactants are ethylene oxide derivatives of alcohols, usually mono-functional primary alcohols or aromatic alcohols (which often have an alkyl substituent), although other alcohol derivatives are also available (e.g. sterol derivatives). Ethoxylated alcohol surfactants have the general formula:

R—(OCH₂CH₂)ₙOH

wherein the group R is the moiety from the original alcohol. For convenience herein, ethoxylated alcohol surfactants are separated into those having an aromatic alcohol (ethoxylated aromatic alcohol surfactants) and those which do not have an aromatic alcohol (ethoxylated non-aromatic alcohol surfactants).

In respect of ethoxylated aromatic alcohol surfactants, suitably the surfactant HLB will be in the range from about 14.0 to about 17.0, in particular from about 14.5 to about 16.5 (such as from 14.5 to less than 15.5, or alternatively between 15.5 and 16.5). Ethoxylated aromatic alcohol surfactants of particular interest are those derived from phenol with an alkyl substituent having between 6 and 12 carbon atoms (which substituent is typically unbranched), e.g. those derived from octylphenol and nonylphenol (in particular nonylphenol). Ethoxylated aromatic alcohol surfactants of use in the present invention will typically contain between 5 and 150 PEG units, suitably between 5 and 40 PEG units, especially between 8 and 25 PEG units, in particular between 10 and 20 PEG units. Exemplary octoxynol surfactants of interest are those having 8 to 29 PEG units, such as 11 to 25 PEG units, especially 15 to 20 PEG units. Exemplary nonoxynol surfactants of interest are those having 8 to 29 PEG units, such as 11 to 25 PEG units, especially 12 to 20 PEG units (e.g. 12 to 16 PEG units). Specific examples of ethoxylated aromatic alcohol surfactants of use in the present invention are octoxynol-12, nonoxynol-15, octoxynol-16 and nonoxynol-20.

In respect of ethoxylated non-aromatic alcohol surfactants, suitably the surfactant HLB will be in the range from about 12.5 to about 17.5, in particular about 13.0 to about 17.0.

Ethoxylated non-aromatic alcohol surfactants include the groups of surfactants known as propylene glycol POE ethers (e.g. alkyl or alkenyl ethers, in particular alkyl) of the general formula:

R—OCH(CH₃)CH₂—(OCH₂CH₂)ₙOH

Ethoxylated non-aromatic alcohol surfactants of particular interest are those derived from alkyl or alkenyl alcohols (typically monofunctional alcohols, e.g. primary alcohols) having between 10 and 24 carbon atoms (which is typically unbranched and may optionally contain 1 or 2 double bonds, such as 1 double bond), e.g. laureth, trideceth, myristeth, ceteth, isoceteth, steareth, isosteareth, oleth and beheneth, or mixtures such as pareth and ceteareth (in particular laureth, ceteth, isoceteth, isosteareth, oleth, C11-15 pareth, C12-13 pareth and ceteareth). A further group of ethoxylated non-aromatic alcohol surfactants of particular interest are those derived from coceth. Ethoxylated non-aromatic alcohol surfactants of use in the present invention will typically contain between 5 and 150 PEG units, suitably between 5 and 50 PEG units, especially between 5 and 40 PEG units, in particular between 8 and 30 PEG units.

One group of ethoxylated non-aromatic alcohol surfactants of use in the present invention are the laureth series having between 5 and 150 PEG units, such as between 8 and 50 PEG units, for example between 8 and 23 PEG units (those having an HLB of 13.1 or greater, such as 13.5 or greater, are of particular interest, for example those having an HLB of 13.1 to 17.5, especially 13.5 to 17.0). Exemplary laureth series ethoxylated non-aromatic alcohol surfactants of interest are those having 10 to 40 PEG units, especially 10 to 25 PEG units. Specific examples of laureth series ethoxylated non-aromatic alcohol surfactants of use in the present invention are laureth-8, laureth-10 and laureth-23 (especially laureth-10 and laureth-23).

Another specific group of ethoxylated non-aromatic alcohol surfactants of use in the present invention are the ceteth series having between 5 and 150 PEG units, such as between 10 and 50 PEG units, for example between 15 and 20 PEG units (those having an HLB of 13.0 or greater, such as 15.5 or greater, are of particular interest, for example those having an HLB of 14.0 to 17.5, especially 15.0 to 16.0). Exemplary ceteth series ethoxylated non-aromatic alcohol surfactants of interest are those having 10 to 40 PEG units, such as 10 to 24 PEG units, especially 10 to 20 PEG units. Specific examples of ceteth series ethoxylated non-aromatic alcohol surfactants of use in the present invention are ceteth-10, ceteth-15 and ceteth-20 (especially ceteth-15 and ceteth-20).

A further specific group of ethoxylated non-aromatic alcohol surfactants of use in the present invention are the oleth series having between 5 and 150 PEG units, such as between 10 and 50 PEG units, for example between 15 and 20 PEG units (those having an HLB of 12.5 or greater, such as 14.2 or greater, are of particular interest, for example those having an HLB of 13.0 to 17.0, especially 14.2 to 16.0). Exemplary oleth series ethoxylated non-aromatic alcohol surfactants of interest are those having 12 to 50 PEG units, such as 12 to 40 PEG units, especially 15 to 30 PEG units. Specific examples of oleth series ethoxylated non-aromatic alcohol surfactants of use in the present invention are oleth-15, oleth-20 and oleth-30 (especially oleth-15 and oleth-20).

Ethoxylated non-aromatic alcohol surfactants of the pareth series (e.g. C11-15 pareth, or alternatively C12-13 pareth) are also of interest, such as those having between 5 and 150 PEG units, such as between 10 and 35 PEG units, for example between 12 and 23 PEG units (those having an HLB of between 14.0 and 17.5, such as those between 14.7 and 16.7, are of particular interest). Exemplary pareth series ethoxylated non-aromatic alcohol surfactants of interest are those having 12 to 30 PEG units. Specific examples of pareth series ethoxylated non-aromatic alcohol surfactants of use in the present invention are C11-15 pareth-12, C11-15 pareth-15, C11-15 pareth-20 and C12-C13 pareth-23 (especially C11-15 pareth-15, C11-15 pareth-20 and C12-C13 pareth-23).

Another specific group of ethoxylated non-aromatic alcohol surfactants of use in the present invention are the ceteareth series having between 5 and 150 PEG units, such as between 10 and 50 PEG units, for example between 20 and 30 PEG units, especially 22 to 28 PEG units (those having an HLB between 15.5 and 17.0, such as those between 15.7 and 16.7, are of particular interest). Specific examples of ceteareth series ethoxylated non-aromatic alcohol surfactants of use in the present invention are ceteareth-20, ceteareth-25 and ceteareth-30 (especially ceteareth-25).

Other ethoxylated non-aromatic alcohol surfactants of use in the present invention include the isoceteth series having between 5 and 150 PEG units, such as between 10 and 50 PEG units, for example between 15 and 25 PEG units (those having an HLB between 14.0 and 17.0, such as those between 15.2 and 16.2, are of particular interest). A specific example of an isoceteth series ethoxylated non-aromatic alcohol surfactant of use in the present invention is isoceteth-20.

Further ethoxylated non-aromatic alcohol surfactants of use in the present invention include the isosteareth series having between 5 and 150 PEG units, such as between 10 and 50 PEG units, for example between 15 and 25 PEG units (those having an HLB between 14.0 and 17.0, such as those between 14.5 and 15.5, are of particular interest). A specific example of an isosteareth series ethoxylated non-aromatic alcohol surfactant of use in the present invention is isosteareth-20.

Another specific group of ethoxylated non-aromatic alcohol surfactants of use in the present invention are the coceth series having between 5 and 150 PEG units, such as between 5 and 50 PEG units, especially 8 to 30 PEG units, for example 10 and 20 PEG units (those having an HLB between 13.0 and 17.0, such as those between 13.5 and 16.5, especially between 14 and 16, are of particular interest). Specific examples of coceth series ethoxylated non-aromatic alcohol surfactants of use in the present invention are coceth-10 and coceth-20.

Propoxylated/ethoxylated ethers covers a number of groups of surfactants including ethoxylated PPG alkyl ethers, ethoxylated PPG ethers and propoxylated POE ethers.

Ethoxylated PPG alkyl ethers have the general formula:

R—(OCH(CH₃)CH₂)ₘ(OCH₂CH₂)ₙOH

wherein R represents an alkyl or alkenyl chain. Typically the R group is an unbranched alkyl of 10 to 22 carbon atoms in length.

Ethoxylated PPG ethers have the general formula:

H(OCH₂CH₂)ₘ(OCH(CH₃)CH₂)ₓ(OCH₂CH₂)ₙOH

Propoxylated POE ethers have the general formula:

H(OCH(CH₃)CH₂)ₘ(OCH₂CH₂)ₓ(OCH(CH₃)CH₂)ₙOH

Polyglyceryl ethers can be prepared by the reaction of an alcohol (e.g. monofunctional) with polyglycerol. Suitably the polyglyceryl chain will be from 2 to 50 units in length. Suitably the alcohol is an alkyl or alkenyl alcohol (e.g. primary alcohols) having between 10 and 24 carbon atoms (which is typically unbranched and may optionally contain 1 or 2 double bonds, such as 1 double bond), e.g. laureth, trideceth, myristeth, ceteth, isoceteth, steareth, isosteareth, oleth and beheneth, or mixtures such as pareth and ceteareth (in particular laureth, ceteth, isoceteth, isosteareth, oleth, C11-15 pareth, C12-13 pareth and ceteareth). Further examples are those derived from coceth. Polyglyceryl ethers may be mono or polyethers.

Sugar ethers are a class of surfactant prepared from the derivatisation of an alcohol (e.g. a monofunctional alcohol) with mono or polysaccharides. Suitably the alcohol is a primary alcohols having between 10 and 24 carbon atoms (which is typically unbranched and may optionally contain 1 or 2 double bonds, such as 1 double bond), e.g. laureth, trideceth, myristeth, ceteth, isoceteth, steareth, isosteareth, oleth and beheneth, or mixtures such as pareth and ceteareth (in particular laureth, ceteth, isoceteth, isosteareth, oleth, C11-15 pareth, C12-13 pareth and ceteareth). Further examples are those derived from coceth. Suitably the number of sugar residues will be from 1 to 10 (e.g. 1 sugar residue). Suitably the mono or polysaccharide is a glycoside.

### Esters

In one embodiment of the invention the surfactant is an ester surfactant.

The broad class of ester surfactants may be separated into a number of sub-classes which include:
ethoxylated carboxylic acids
ethoxylated glycerides
polyglyceryl esters
sugar esters

In one embodiment of the invention the ester surfactant is an ethoxylated carboxylic acid. In a second embodiment of the invention the ester surfactant is an ethoxylated glyceride. In a third embodiment of the invention the ester surfactant is a polyglyceryl ester. In a fourth embodiment of the invention the ester surfactant is a sugar ester.

Ethoxylated carboxylic acid surfactants are ethylene oxide derivatives of carboxylic acids, usually mono-functional primary alkyl or alkenyl acids. Ethoxylated carboxylic acid surfactants have the general formula:

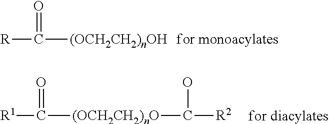

wherein the group R is the moiety from the original acid (in diacylates, R¹ and R² both typically represent the same moiety).

In respect of ethoxylated carboxylic acid surfactants, suitably the surfactant HLB will be in the range from about 12.5 to about 17.5, in particular about 13.0 to about 17.0. Ethoxylated carboxylic acid surfactants of particular interest are those derived from alkyl or alkenyl acids (typically monofunctional acids, e.g. primary acids) having between 10 and 24 carbon atoms (which is typically unbranched and may optionally contain 1 or 2 double bonds, such as 1 double bond), e.g. laurate, myristate, palmitate, stearate and oleate (in particular stearate), or mixtures thereof. Ethoxylated carboxylic acid surfactants of use in the present invention will typically contain between 5 and 150 PEG units, suitably between 5 and 50 PEG units, especially between 10 and 45 PEG units, in particular between 20 and 40 PEG units.

In one embodiment of the invention the ethoxylated carboxylic acid surfactant is substantially monoacylated. In a second embodiment of the invention the ethoxylated carboxylic acid surfactant is substantially diacylated. In a third embodiment of the invention the ethoxylated carboxylic acid surfactant is a mixture of the ethoxylated carboxylic acid surfactants having varying degrees of acylation (e.g. averaging 1.5 acyl units).

One group of ethoxylated carboxylic acid surfactants of use in the present invention are the stearate series having between 5 and 150 PEG units, such as between 10 and 50 PEG units, for example between 20 and 40 PEG units (those having an HLB between 15.5 and 17.5, such as those between 16.0 and 16.9, are of particular interest). Specific examples of stearate series ethoxylated carboxylic acid surfactants of use in the present invention are PEG-20 stearate and PEG-40 stearate.

Ethoxylated glycerides are of the general formula:

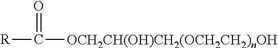

where R is the moiety from the carboxylic acid. Ethoxylated glyceride surfactants of particular interest are those derived from alkyl or alkenyl acids (typically monofunctional acids, e.g. primary acids) having between 10 and 24 carbon atoms (which is typically unbranched and may optionally contain 1 or 2 double bonds, such as 1 double bond), e.g. laurate, myristate, palmitate, stearate and oleate, or mixtures thereof. Ethoxylated glyceride surfactants of use in the present invention will typically contain between 5 and 150 PEG units, suitably between 5 and 50 PEG units, especially between 10 and 45 PEG units.

Polyglyceryl esters can be prepared by the reaction of a carboxylic acid with polyglycerol. Suitably the polyglyceryl chain will be from 2 to 50 units in length. Suitably the carboxylic acid is an alkyl or alkenyl acid (typically monofunctional acids, e.g. primary acids) having between 10 and 24 carbon atoms (which is typically unbranched and may optionally contain 1 or 2 double bonds, such as 1 double bond), e.g. laurate, myristate, palmitate, stearate and oleate, or mixtures thereof. Polyglyceryl esters may be mono or polyesters.

Sugar esters can be divided into two main groups, the sorbitan esters and the non-sorbitan esters.

Sorbitan/sorbitol esters are based around a sorbitan/sorbitol core which is derivatised by reaction with a carboxylic acid. The simplest sorbitan ester surfactants are acylated, generally being monoacylated on average, containing only the hydrophilic sorbitan ring and the hydrophobic moiety from an alkyl or alkenyl acid. Typically, the alkyl or alkenyl acid (typically monofunctional acids, e.g. primary acids) has between 10 and 24 carbon atoms (which is typically unbranched and may optionally contain 1 or 2 double bonds, such as 1 double bond), e.g. laurate, myristate, palmitate, stearate and oleate, or mixtures thereof (in particular laurate and oleate, especially laurate). Such acylated sorbitan esters generally have a very low HLB which precludes them from being of use in the present invention. However, acylated sorbitan esters can be further derivatised by ethoxylation to provide PEG sorbitan esters which are more hydrophilic and have higher HLB numbers.

PEG sorbitan esters typically contain between 5 and 150 PEG units, such as between 10 and 50 PEG units, especially 10 to 30 PEG units, in particular 15 to 25 PEG units, such as 20 PEG units (those having an HLB between 15.7 and 17.5, such as those between 16.2 and 17.2, are of particular interest. Exemplary oleate and laurate series PEG sorbitan esters of interest are those having 10 to 30 PEG units, such as 15 to 25 PEG units. A specific example of a PEG sorbitan ester of use in the present invention is polysorbate 20.

Non-sorbitan sugar esters form an analogous group to the sorbitan esters, having a sugar core (e.g. sucrose, glucose or methyl glucose, in particular sucrose or glucose, especially sucrose) which is derivatised by reaction with a carboxylic acid. Typically the carboxylic acid is an alkyl or alkenyl acid (typically monofunctional acids, e.g. primary acids) having between 6 and 22 carbon atoms (which is typically unbranched and may optionally contain 1 or 2 double bonds, such as 1 double bond), e.g. octanoate, decanoate, laurate, myristate, palmitate, stearate and oleate (in particular decanoate, laurate and myristate), or mixtures thereof. Sugar ester surfactants may be mono or polyacylated (or a mixture of such), typically those monoacylated or diacylated on average are of particular interest, especially monoacylated. Specific examples of sugar ester surfactants of use in the present invention include sucrose laurate, sucrose myristate and decyl glucoside.

Non-sorbitan sugar esters can be further derivatised to provide PEG non-sorbitan sugar ester surfactants, typically containing between 5 and 150 PEG units, such as between 10 and 50 PEG units.

Suitably, when the surfactant is a sugar ester, the sugar ester is a PEG sorbitan ester or a non-sorbitan sugar ester.

### Ionic Surfactants

Ionic surfactants are a further broad class of surface active agents which may be used in the present invention.

Ionic surfactants include:
cationic surfactants
anionic surfactants
amphoteric surfactants

Cationic surfactants are those having a positive charge in aqueous solution at neutral pH. One series of cationic surfactants of particular interest is the PEG alkyl amines.

PEG alkyl amines have the following general structure:

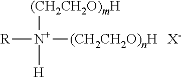

wherein R is typically an alkyl or alkenyl group (X⁻ is a counter anion (typically a halide, such as chloride). PEG alkyl amines of particular interest have between 6 and 22 carbon atoms (which is typically unbranched and may optionally contain 1 or 2 double bonds, such as 1 double bond), e.g. being derived from decylamine, laurylamine, myristylamine, cetylamine, stearylamine and oleylamine, or mixtures such as cocamine. The total number of PEG units (i.e. m+n) typically being from 2 to 50, such as 2 to 30, in particular 2 to 15. Exemplary cocamine series PEG alkyl amines of interest are those having 2 to 30 PEG units, such as 2 to 25 PEG units, for example 5 to 10 PEG units. Specific examples of PEG alkyl amines of use in the present invention include PEG-5 cocamine and PEG-15 cocamine.

Anionic surfactants are those having a negative charge in aqueous solution at neutral pH.

Anionic surfactants include, for example, the alkyl and alkenyl acids, amino acid amides, esters of alpha-hydroxycarboxylic acids and a range of other materials such as sulphate or phosphate based surfactants. Alkyl and alkenyl acids may be typically expected to have insufficient hydrophilicity for use in the present invention. Anionic surfactants of the amino acid amide group are of particular interest.

Anionic amino acid amide surfactants are amino acids (i.e. non-basic amino acids) which have been acylated by reaction with a carboxylic acid. Suitably the amino acid is glutamic acid or glycine, although a number of commercial surfactants are available based on plant derived mixtures of amino acids (e.g. wheat and oat). Typically the carboxylic acid is an alkyl or alkenyl acid (typically monofunctional acids, e.g. primary acids) having between 6 and 22 carbon atoms (which is typically unbranched and may optionally contain 1 or 2 double bonds, such as 1 double bond), e.g. lauroyl and stearoyl, or mixtures such as cocoyl (in particular lauroyl and cocoyl). Specific examples of amino acid amide surfactants of use in the present invention include sodium lauroyl glutamate, sodium cocoyl glycinate, sodium cocoyl methyl taurate, sodium cocoyl glutamate, disodium cocoyl glutamate, sodium lauryl wheat amino acids, potassium lauryl wheat amino acids, sodium lauryl oat amino acids and sodium cocoyl apple amino acids (especially sodium lauroyl glutamate, sodium cocoyl glycinate, sodium cocoyl glutamate, potassium lauryl wheat amino acids and sodium lauryl oat amino acids).

Another anionic amino acid derived surfactant is surfactin (Aminofect).

Esters of alpha-hydroxycarboxylic acids are materials wherein the hydroxyl function of an alpha-hydroxycarboxylic acid (e.g. lactic acid) is esterified with a carboxylic acid, typically the carboxylic acid is an alkyl or alkenyl acid (typically monofunctional acids, e.g. primary acids) having between 6 and 22 carbon atoms (which is typically unbranched and may optionally contain 1 or 2 double bonds, such as 1 double bond), e.g. lauryl. Such materials generally have relatively low HLB values, therefore would not typically be expected to be of use in the present invention.

Phosphate based surfactants include groups such as the alkyl and alkenyl phosphates (e.g. cetyl phosphate and such like). Other phosphate based surfactants are the PPG ethoxylated alkyl phosphates (e.g. PPG-5 ceteth-10 phosphate), wherein the number of PPG units will typically vary from 2 to 20, the number of PEG units typically vary from 5 to 50 and the aliphatic ether will be derived from an alkyl or alkenyl alcohol (typically monofunctional alcohols, e.g. primary alcohols) having between 10 and 24 carbon atoms (which is typically unbranched and may optionally contain 1 or 2 double bonds, such as 1 double bond) such as ceteth.

Sulphate based surfactants include sodium cholate and sodium deoxycholate. Another sulphate based surfactant is sodium lauryl sulphate. Sulphate based surfactants such as sodium cholate, sodium deoxycholate and sodium lauryl sulphate are highly potent surfactants and are recognised as irritants.

Zwiterionic or amphoteric surfactants are those having a positive and a negative charge in aqueous solution at neutral pH. Amphoteric surfactants include amino acid amide surfactants wherein the amino acid is a basic amino acid and which has been acylated by reaction with a carboxylic acid. Typically the carboxylic acid is an alkyl or alkenyl acid (typically monofunctional acids, e.g. primary acids) having between 6 and 22 carbon atoms (which is typically unbranched and may optionally contain 1 or 2 double bonds, such as 1 double bond).

Other amphoteric surfactants include materials such as cocamidopropyl betaine, wherein a betaine hydrophile is attached to a hydrophobic chain which incorporates an amide linkage.

Amphoteric polymeric surfactants include amphipol A8-35 (see Gohon Y et al Analytical Biochemistry 2004 334:318-334; Pocanschi C L et al Biochemistry 2006 45:13954-13961).

### Polymer Surfactants

In addition to the surfactants outlined above, it will be clear to those skilled in the art that polymers or copolymers with a suitable balance of hydrophilic/lipophilic blocks (i.e. equivalent to having a suitable HLB value) could also be envisaged which would be suitable for use as surfactants in the present invention.

Non-biodegradable polymeric surfactants which may be of use in the present invention include non-alternating co-polymers of hydrolysed maleic anhydride and alkyl vinyl ethers in which the ratio of monomer units is such that the polymer has the correct HLB value by virtue of the charge on the carboxylic acid groups under the conditions of use (e.g. pH between 5.5-8.5) and the proportion (e.g. about 2:1, 3:1 or 4:1, based on an excess of hydrophobic groups) and type of hydrophobic groups present (e.g. propyl or butyl).

Biodegradable polymeric surfactants include polyester co-polymers of mandelic and malic acid in which the ratio of monomer units is such that the polymer has the correct HLB value by virtue of the charge on the carboxylic acid groups on the malic acid units under the conditions of use (e.g. pH between 5.5-8.5) and the proportion (e.g. about 2:1, 3:1 or 4:1, based on an excess of hydrophobic groups) of the hydrophobic groups provided by the mandelic acid units.

Particularly suitable polymeric surfactants are copolymers of styrene and maleic acid, prepared by hydrolysis of copolymers of styrene and maleic anhydride.

### Alternating Copolymers of Styrene and Maleic Anhydride

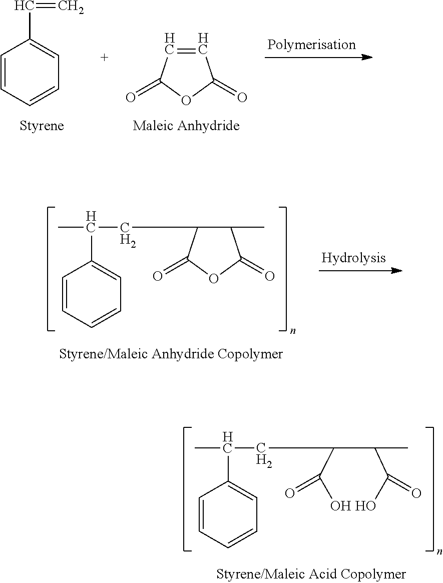

Alternating copolymers of styrene and maleic acid (i.e. hydrolysed styrene/maleic anhydride polymers) have a pKₐ value in the region of 3.75-4.0 (Sugai, S and Ohno, N Biophys. Chem. 1980 11:387-395), the pKₐ for the individual acid functions being approximately 1.97 and 6.24. Preparation of clear solutions, and hence macromolecular assemblies, requires a lowering of the pH to between 3-5. Such pH levels are not generally suitable for compositions which are to be applied to the body. Although the pH of these alternating copolymer formulations may be raised after the formation of the polymer/lipid complex, such adjustment leads to instability, which may be observed as a loss of clarity over time as the macromolecular assemblies degrade.

In a one aspect of the present invention the copolymer of styrene and maleic acid is alternating. Suitably, the copolymer of styrene and maleic acid is non-alternating (e.g. wherein the ratio of styrene to maleic acid monomer units is greater than 1:1).

Monomer ratios stated for polymers are defined on the basis of the number of each monomer unit in the polymer, for example, a ratio of styrene and maleic anhydride of 3:1 indicates that there are three styrene monomer units for each maleic anhydride monomer unit in the polymer chain. It will be understood that the stated monomer ratios are averages and, as a result of the uncertainty in polymerisation reactions, do not necessarily represent the exact ratio for any specific polymer chain. Typically greater than 50%, in particular greater than 75% and especially greater than 90% (on a weight to weight basis) and suitably all of the polymer chains will have a monomer ratio which is within 50%, such as within 35%, suitably 25% (for example within 15%), more particularly within 10% and especially within 5% of the stated value. For example, a ratio of styrene and maleic anhydride of 3:1 with 10% variation covers 3.3:1 to 2.7:1. Free-radical-initiated copolymerisation of styrene and maleic anhydride is an extremely well characterised polymerisation reaction (Trivedi, B C and Culbertson, B M Maleic Anhydride, Plenum (1982), ISBN 0306409291). The reactivity ratios, r₁ and r₂, for any monomer pair may be used as an index for evaluating the alternating frequency in copolymerisation reactions. Ideal (i.e. random) copolymerisation conditions exist when r₁, r₂ and r₁r₂ are equal to 1. Where r₁, r₂ and r₁r₂ tend to zero, the degree of alternation increases. The reactivity ratios r₁ and r₂ of styrene (monomer 1) with maleic anhydride (monomer 2) are 0.097 and 0.001 respectively (Fried, J Polymer Science and Technology, 2^{nd} Ed, Prentice Hall (2003), ISBN 0130181684), indicating that although both monomers preferentially react with the other, styrene is significantly less discriminating than maleic anhydride. Consequently, the sequence distribution within a copolymer of styrene and maleic anhydride depends upon the monomer feed composition and the resulting copolymers can differ from 1:1 alternation. In cases where the ratio of styrene to maleic anhydride is greater than 1:1 (for example 2:1, 3:1 or 4:1) an increasing number of styrene-styrene sequences are present.

Styrene/maleic anhydride copolymers are conveniently prepared by a precipitation process, typically in an aromatic hydrocarbon solvent, for example toluene or dichlorobenzene. Polymerisation may be initiated using free-radical initiators, for example AIBN (azoisobutyronitrile) and the molecular weight may be controlled by the use of end-capping agents such as highly alkylated aromatic hydrocarbons, for example p-cymene. The ratio of monomers in the polymer may be controlled by variation of the feed composition, and may be determined by means known to those skilled in the art, for example by titration to determine maleic acid content of the hydrolysed polymer.

Styrene/maleic acid copolymers of use in the present invention will typically have an average molecular weight (M_{w}) of less than 500,000 daltons, especially less than 150,000 daltons, in particular less than 50,000 daltons and suitably less than 20,000 daltons (for example 1,500 to 15,000 daltons). M_{w}/Mₙ (Mₙ being the number average molecular weight) indicates the polydispersity, and will typically be less than 5, especially less than 4, in particular less than 3 and suitably less than 2 (for example less than 1.5). Polymers should generally be of sufficient length such that they may demonstrate the ability to hypercoil, but are suitably not so long as to introduce difficulties with viscosity as a result of interchain interactions.

A number of blocky styrene/maleic anhydride copolymers are commercially available from Sartomer Inc., and are sold under the tradenames SMA2000, SMA3000 and SMA4000. In the case of SMA2000, SMA3000 and SMA4000 the ratio of styrene to maleic anhydride is to 2:1, 3:1 and 4:1 respectively. In these instances, the styrene forms an increasing number of short blocks as the styrene content is increased. SMA2000, SMA3000 and SMA4000 are available as powder, flake or ultrafine powder preparations. Typical molecular weights for SMA2000 are M_{w} 7,500 (Mₙ 2,700); for SMA3000 are M_{w} 9,500 (Mₙ 3,050) and for SMA4000 are M_{w} 11,000 (Mₙ 3,600) as assessed by gel permeation chromatography (GPC).

Styrene/maleic anhydride copolymers must be hydrolysed for use in the present invention, and such hydrolysed polymers may optionally be used in the form of a salt. The polymers may be hydrolysed by a number of means, for example by reflux in aqueous solution, suitably in the presence of a strong base such as sodium hydroxide. Partially hydrolysed styrene/maleic anhydride copolymers may also be of use in the present invention, however, in aqueous solution these are likely to hydrolyse further and for reasons of stability, fully hydrolysed polymer is typically used.

Certain salts of hydrolysed styrene/maleic anhydride copolymers are available commercially, for example, SMA3000HNa is a sodium salt of hydrolysed SMA3000, SMA3000HK is a potassium salt of hydrolysed SMA3000, and SMA4000HNa is a sodium salt of hydrolysed SMA4000. Other salt forms are also available commercially, such as the ammonium salt. Although suitable for use in the present invention, ammonium salts are generally less desirable in cosmetic and pharmaceutical applications due to their associated odours.

Commercial grades of the styrene/maleic anhydride copolymers, as supplied for industrial uses, may contain monomer, end-capping agent residuals and initiator residuals (e.g. maleic anhydride, styrene, cumene and acetophenone), which residuals are generally undesirable in compositions for use in personal care, cosmetic, pharmaceutical or biomedical products. Residual impurities may be removed or reduced in quantity by means known to those skilled in the art, such techniques include but are not limited to the selective solvation of the residual components into alcohols (for example methanol, ethanol or isopropanol) or into chlorinated solvents (for example chloroform or dichloromethane).

Hydrolysed styrene/maleic anhydride copolymers, i.e. styrene/maleic acid, and salts thereof (e.g. cosmetically and pharmaceutically acceptable salts, such as alkali metal salts, for example potassium or sodium), of use in the present invention will typically have a monomer ratio of styrene to maleic acid of greater than 1:1, in particular greater than 1.2:1, especially greater than 1.5:1, suitably greater than 2.5:1; while additionally typically having a ratio of styrene to maleic acid of less than 4.5:1, especially less than 3.5:1. Exemplary monomer ratios of use in the present invention include: 2:1, 3:1 and 4:1, suitably 2:1 or 3:1. In one embodiment of the invention the ratio of styrene and maleic acid monomer units is about 2:1. In a second embodiment of the invention the ratio of styrene and maleic acid monomer units is about 3:1.

In one embodiment of the invention the copolymer of styrene and maleic acid (or salt thereof) has an average molecular weight in the range 4,500 to 12,000 and a ratio of styrene to maleic acid of about 2:1, 3:1 or 4:1, in particular about 2:1 or about 3:1.

Although formulations for repeated application to the skin may be slightly acidic, typically being in the pH 5.0-7.5 range, particularly pH 5.5-7.5, formulations for application to other sites, or for internal administration, should typically be maintained around pH 6.5-7.5. Formulations specifically for application to the eye are ideally in the range pH 7.1-7.8, more particularly pH 7.3-7.6 (Carney, L G and Hill, R M Arch. Ophthalmol. 1976 94(5):821-824). Styrene/maleic acid copolymers with a monomer ratio of styrene to maleic acid of greater than 1:1 and less than 4.5:1 may interact with lipids to form stable macromolecular complexes at pH levels suitable for physiological use (e.g. within the ranges described above). It should be noted that specific embodiments may not necessarily demonstrate stable polymer and lipid macromolecular assemblies across the entire pH ranges specified.

### Particularly Suitable Surfactants

Suitably the surfactant will be an ethoxylated alcohol ether surfactant, an ethoxylated carboxylic acid surfactant, a sugar ester surfactant, a PEG alkyl amine surfactant, anionic amino acid amide surfactant or surfactin.

Specific examples of surfactants of use in the present invention include octoxynol-12, nonoxynol-15, octoxynol-16, nonoxynol-20, laureth-8, laureth-10, laureth 23, ceteth-10, ceteth-15, ceteth-20, oleth-15, oleth-20, C11-15 pareth-12, C11-15 pareth-15, C11-15 pareth-20, C11-15 pareth-20, C12-C13 pareth-23, ceteareth-20, ceteareth-25, ceteareth-30, isoceteth-20, isosteareth-20, PEG-20 stearate, PEG-40 stearate, polysorbate 20, sucrose laurate, sucrose myristate, decyl glucoside, PEG-5 cocamine, PEG-15 cocamine, sodium lauroyl glutamate, sodium cocoyl glycinate, sodium cocoyl glutamate, disodium cocoyl glutamate, potassium lauryl wheat amino acids, sodium lauryl oat amino acids, sodium lauryl wheat amino acids, sodium cocoyl apple amino acids, sodium cocoyl methyl taurate and surfactin; especially octoxynol-12, nonoxynol-15, octoxynol-16, nonoxynol-20, laureth-10, laureth 23, ceteth-10, ceteth-15, ceteth-20, oleth-15, oleth-20, C11-15 pareth-12, C11-15 pareth-15, C11-15 pareth-20, C11-15 pareth-20, C12-C13 pareth-23, ceteareth-20, ceteareth-25, isoceteth-20, isosteareth-20, PEG-20 stearate, polysorbate 20, sucrose laurate, sucrose myristate, decyl glucoside, PEG-5 cocamine, PEG-15 cocamine, sodium lauroyl glutamate, sodium cocoyl glycinate, sodium cocoyl glutamate, disodium cocoyl glutamate, potassium lauryl wheat amino acids, sodium lauryl oat amino acids, sodium lauryl wheat amino acids, sodium cocoyl apple amino acids, sodium cocoyl methyl taurate and surfactin; in particular octoxynol-12, nonoxynol-15, octoxynol-16, nonoxynol-20, laureth-10, laureth 23, ceteth-10, ceteth-15, ceteth-20, oleth-15, oleth-20, C11-15 pareth-15, C11-15 pareth-20, C11-15 pareth-20, C12-C13 pareth-23, ceteareth-25, isoceteth-20, polysorbate 20, sucrose laurate, sucrose myristate, decyl glucoside, PEG-5 cocamine, PEG-15 cocamine, sodium lauroyl glutamate, sodium cocoyl glycinate, sodium cocoyl glutamate, potassium lauryl wheat amino acids, sodium lauryl wheat amino acids, sodium lauryl oat amino acids and surfactin. Additional examples include coceth-10 and coceth-20.

The suitability of a particular surfactant or surfactant mixture for use in the present invention may be determined by those skilled in the art by routine experimentation based on the guidance provided herein.

### Lipid

The term lipid is well known in the art. The lipid of use in the present invention will typically be selected from phospholipids, ceramides, sphingomyleins, phosphatidic acids, cardiolipins, lysophospholipids, plasmalogens, phosphosphingolipids and mixtures thereof.

Phospholipids (for example phosphatidylcholine, phosphatidylethanolamine, phosphatidylglycerol, phosphatidylinositol, phosphatidylserine and mixtures thereof) have a polar head group (which in a membrane aligns towards the aqueous phase) and two hydrophobic tail groups (which in a bilayer membrane associate to form a hydrophobic core). The hydrophobic tail groups will typically be in the form of acyl esters, which may vary both in their length (for example from 8 to 26 carbon atoms, especially 10 to 20 carbon atoms) and their degree of unsaturation (for example one, two or three double bonds, especially one double bond). Generally, the two hydrophobic tail groups are identical, though they need not be so.

Lipids of use in the present invention may be of natural or synthetic origin, and may be: a single pure component (e.g. at least 80% pure, especially at least 90% pure, in particular at least 95% pure and suitably at least 99% pure on a weight basis); a single class of lipid components (for example a mixture of phosphatidylcholines, or alternatively, a mixture of lipids with a conserved acyl chain type) or may be a mixture of many different lipid types.

In one embodiment of the invention the lipid is a single pure component.

Pure lipids are generally of synthetic or semi-synthetic origin. Examples of pure lipids of use in the present invention include pure phosphatidylcholines (for example, DMPC, DLPC, DPPC and DSPC, in particular DLPC and DPPC, especially DLPC) and phosphatidylglycerols (for example DPPG), suitably phosphatidylcholines. The use of pure lipids is desirable due to their clearly defined composition, however, they are generally prohibitively expensive for many commercial applications.

In a second embodiment of the invention the lipid is a mixture of components.

Mixtures of lipids of use in the present invention may be of natural origin, obtained by extraction and purification by means known to those skilled in the art. Lipid mixtures of natural origin are generally significantly cheaper than pure synthetic lipids. Naturally derived lipids include lipid extracts from egg or soy, which extracts will generally contain lipids with a mixture of acyl chain lengths, degrees of unsaturation and headgroup types. Lipid extracts of plant origin may typically be expected to demonstrate higher levels of unsaturation than those of animal origin. It should be noted that, due to variation in the source, the composition of lipid extracts may vary from batch to batch. Hydrogenated lipids are less prone to peroxidation due to the absence of unsaturation, typically have less coloration and have lower odour.

Lipid mixtures may also be prepared by the combination of pure lipids, or by the combination of one lipid extract with either other lipid extracts or with pure lipids. The preparation of lipid mixtures by the combination of lipid extracts and/or pure lipids is of particular relevance to compositions for use in the analysis of membrane proteins/peptides and their interactions with other agents, wherein it is highly desirable to control the lipid constituents such that the natural environment is closely mimicked.

Suitably, a lipid extract of use in the present invention will comprise at least 50% phospholipids by weight (for example, phosphatidylcholines and phosphatidylethanolamines), especially at least 55% phospholipids by weight, in particular at least 60% phospholipids by weight (such as 75% or 90%).

In one embodiment of the invention the lipid mixture is a lipid extract containing at least 50%, such as at least 60%, especially at least 75% and suitably at least 90% by weight of phospholipids of a single headgroup type (e.g. phosphatidylcholines). In a second embodiment of the invention particular lipid extracts may be of particular interest due to their relatively cheap cost. In a third embodiment of the invention lipid extracts of particular interest are those which result in solutions of highest clarity. In a fourth embodiment of the invention the lipid is a lipid mixture having a conserved acyl chain length (e.g. at least 50%, such as at least 60%, especially at least 75% and suitably at least 90% by weight), for example 12 (e.g. lauryl), 14 (e.g. myristyl), 16 (e.g. palmityl) or 18 (e.g. stearyl or alternatively oleyl) carbons atoms in length, in particular 12-16 carbon atoms (such acyl chains optionally having one, two or three double bonds, though suitably being fully saturated). In another embodiment of the invention the lipid is a lipid mixture which is hydrogenated (i.e. the acyl chains are fully saturated). In a further embodiment of the invention the lipid mixture is a lipid extract of plant origin (e.g. soy). In another embodiment of the invention the lipid mixture is a lipid extract of animal origin (e.g. egg).

Exemplary lipid extracts of use in the present invention include: Epikuron 200, Epikuron 200SH, Epikuron 145V, Epikuron 130P, Emulmetik 950, Emulmetik 900 and Emulmetik 300 available from Degussa Texturant Systems UK Ltd/Cargill; S 75, S 100, S PC and SL 80 available from Lipoid GmbH; Phospholipon® LPC 20H, Phospholipon® 90 H, Phospholipon® 80 H, and Phospholipon® 90 NG available from Phospolipid GmbH/Lipoid GmbH; EMULTOP® IP and EMULPUR® IP available from Lucas Meyer (Degussa Texturant Systems UK Ltd). A further lipid extract for use in the present invention is Vav S-70 available from VAV Life Sciences Pvt. Ltd., India.

One suitable lipid extract is derived from soy and comprises: at least 92% phosphatidyl cholines, a maximum of 3% lyso-phosphatidyl cholines and a maximum of 2% oils; of which 14-20% of the acyl chains are palmityl, 3-5% stearyl, 8-12% oleic, 62-66% linoleic and 6-8% linolenic. A second suitable lipid extract is derived from soy and comprises: at least 90% hydrogenated phosphatidyl cholines, a maximum of 4% hydrogenated lyso-phosphatidyl cholines and a maximum of 2% oils and triglycerides; of which at least 80% of the acyl chains are stearyl and at least 10% are palmityl.

The lipid, or lipid mixture, of use in the present invention will typically be membrane forming.

Those skilled in the art will recognise that lipid mixtures of use in the invention may comprise non-membrane forming lipid components (e.g. cholesterol). In some circumstances lipid mixtures of use in the invention may be a mixture of only non-membrane forming lipids which in combination demonstrate membrane forming ability.

For cosmetic and pharmaceutical applications typically the lipid (for example the pure lipid or the lipid mixture) is one which has been approved for use in cosmetic and/or pharmaceutical applications as appropriate.

Suitably the lipid is a pure lipid, a plant derived lipid extract or an egg derived lipid extract (especially a pure lipid or a plant derived lipid extract).

The suitability of a particular pure lipid or lipid mixture for use in the present invention may be determined by those skilled in the art by routine experimentation based on the guidance provided herein.

### Macromolecular Assemblies

The presence of a macromolecular assembly (an association of individual surfactant and lipid molecules within a macromolecular structure which is not maintained by covalent bonding), also referred to herein as a macromolecular complex, may be confirmed by a number of means available to those skilled in the art for the determination of particle size, for example, electron microscopy (such as used in Tonge, S R and Tighe, B J Advanced Drug Delivery Reviews 2001 53:109-122 for macromolecular assemblies incorporating alternating styrene/maleic acid copolymers), laser diffraction techniques and such like. A particularly suitable method for the determination of particle size is dynamic light scattering, with instrumentation available from Malvern Instruments, UK (e.g. Malvern Zetasizer Nano ZS).

Without being limited by theory, it is believed that the macromolecular assemblies of use in the present invention are bilayer discs (as opposed to thread/tube-like micelles or conventional mixed micelles) the bilayer discs being a stable intermediate state between vesicles and mixed micelles. The surfactants are believed to act as ‘lipid chaperones’, arranging the lipid bilayers into nanostructured assemblies of a defined size. Although the precise structure of the macromolecular assemblies of the present invention is of academic interest, it is the surprising beneficial properties exhibited which are of more general interest.

In practice the formation of the macromolecular assemblies will often be visible to the naked eye, through changes in solution clarity. For example, when a cloudy emulsion of styrene/maleic acid polymer and lipid is prepared at relatively high pH (such that the polymer is highly charged and most likely in the form of an extended chain), and the pH is then subsequently lowered to a level where the hydrophilic/hydrophobic balance in the polymer chain is suitable for the formation of macromolecular assemblies (this pH level may be referred to as the critical pH) a noticeable solubilisation of lipid may be seen to occur which, depending on the quantities and exact nature of the individual components present, results in a marked partial or complete clearing of the mixture. The critical pH refers to the pH level below which macromolecular assemblies may form. Styrene/maleic acid copolymers have different critical pH values depending upon their specific monomer ratios, the greater the styrene content the higher the critical pH. Once formed, the pH of a solution containing macromolecular assemblies may be raised above the critical pH, although macromolecular assemblies are generally not stable under such conditions and will degrade over time (substantial increases over the critical pH typically result in a more rapid degradation). pH levels which are substantially below the critical pH may also cause the macromolecular assemblies to degrade, as the hydrophobicity of the polymer chains may reach a level where the polymer is no longer soluble in water.

The macromolecular assemblies will typically be of less than 100 nm in diameter, such as less than 75 nm in diameter, especially less than 60 nm in diameter, such as less than 50 nm in diameter (e.g. less than 30 nm). The diameter of macromolecular assemblies may readily be determined by means known to those skilled in the art. Suitably, at least 50%, such as at least 60%, especially at least 70%, in particular at least 80% and most suitably at least 90% (such as at least 95%) of the macromolecular assemblies have the specified diameter. Suitably, the macromolecular assemblies of the present invention will be of at least 5 nm in diameter, such as at least 6 nm in diameter, especially at least 7 nm in diameter, in particular at least 8 nm in diameter (e.g. at least 9 nm, or at least 10 nm). Suitably the macromolecular assemblies of the present invention will be of 6-75 nm in diameter, in particular 7-60 nm in diameter, such as 8-50 nm in diameter.

Those skilled in the art will understand that the term diameter can be applied to non-spherical particles. For bilayer discs the term diameter refers to the disc diameter. For thread/tube-like micelles the term diameter applies to the ‘effective diameter’ when the sizing technique applied is unable to distinguish between different morphologies (see for example Walter et al Biophysics Journal 1991 60:1315-1325, where tube-like micelles of ca. 100 to 300 nm in length and 3 to 5 nm in diameter, are said to compare well to an ‘effective particle size’ of around 16 nm). In one embodiment of the invention the particle size is determined by laser diffraction. In a second embodiment of the invention the particle size is determined by electron microscopy. In a third embodiment of the invention the particle size is determined by neutron scattering.

When the macromolecular assembly particle size is determined by laser diffraction (e.g. by dynamic light scattering), suitably it will be performed using a Malvern Zetasizer. In such cases the principal particle size detected in compositions of the invention will typically be of less than 100 nm in diameter, such as less than 75 nm in diameter, especially less than 60 nm in diameter, such as less than 50 nm in diameter (e.g. less than 30 nm). Suitably, the principal particle size will be of at least 5 nm in diameter, such as at least 6 nm in diameter, especially at least 7 nm in diameter, in particular at least 8 nm in diameter (e.g. at least 9 nm, or at least 10 nm). Suitably, the intensity of the principal particle size will be at least 50%, such as at least 60%, especially at least 70%, in particular at least 80% and most suitably at least 90% (such as at least 95%). The polydispersity index will suitably be less than 0.7, especially less than 0.6, in particular less than 0.5, such as less than 0.4. Suitably the principal particle size will be will be of 6-75 nm in diameter, in particular 7-60 nm in diameter, such as 8-50 nm in diameter.

Knowles T et al Journal of the American Chemical Society 2009 131(22):7484-7485 includes examples of TEM being applied to investigate the size of macromolecular assemblies constructed from styrene/maleic acid copolymer and lipid.

### Clarity

Clarity provides a convenient and ready means for determining that a solution contains particles generally having a small size and a low size dispersion. Changes in clarity over time can provide an indication of particle size instability.

The clarity of a solution may be determined by methods known to those skilled in the art, for example, through the use of a turbidity meter, such as those provided by Orbeco-Helling or Hach-Lange. Turbidity may be based on a number of standard units, such as nephelometric turbidity units (NTU), which are directly interchangeable with formazin nephelometric units (FNU).

By the term “clear”, when used herein in respect of solutions, is meant a solution with a turbidity reading of less than 150 FNU, especially less than 100 FNU, in particular less than 75 FNU, suitably less than 50 FNU A clarity of less than 75 FNU will typically be indicative of a particle size of less than 100 nm. Suitably aqueous solutions of formulations according to the invention will be clear.

Colourless solutions are those that transmit light without absorbance of any particular visible wavelength. Clear solutions may be coloured where they contain a component which absorbs light within the visible range.

### Stability

The terms “stable”, and where appropriate “stability”, may be used to refer to the physical or chemical stability of a preparation.

Physical stability relates to the ability of a formulation to maintain in its original form over a period of time (i.e. the macromolecular assemblies do not degrade—the particle size characteristics remaining essentially unchanged over a given period of time at a particular temperature). For example, a formulation according to the present invention will suitably be physically stable for a period of at least one week, at least one month or ideally at least six months at a temperature of about 4 degrees centigrade or about 25 degrees centigrade.

As such, a stable solution is one in which: the particle size remains within a defined size limit as may be required for a particular use, for example: the principal particle size detected will remain less than 100 nm in diameter, such as less than 75 nm in diameter, especially less than 50 nm in diameter, such as less than 30 nm in diameter (e.g. less than 20 nm); the intensity of the principal particle size will consistently be at least 50%, such as at least 60%, especially at least 70%, in particular at least 80% and most suitably at least 90% (such as at least 95%); and the polydispersity index will suitably remain less than 0.7, especially less than 0.6, in particular less than 0.5, such as less than 0.4. over a period of time (for example, at least one hour, such as at least one day, especially at least one week, in particular at least one month and suitably at least six months) when stored at constant temperature (for example, at 4° C., suitably at 25° C.).

Chemical stability relates to ability of a formulation to maintain its original constitution. Lipid components, certain surfactants and BT itself may all be subject to degradation over time—leading to undesirable consequences such as discolouration and importantly a loss of BT activity. A formulation according to the present invention will suitably be chemically stable for a period of at least one week, at least one month or ideally at least six months at a temperature of about 4 degrees centigrade or about 25 degrees centigrade. The effectiveness of the BT in formulations of the invention can be quantified in assays and should not deteriorate by more than 20% (i.e. at least 80% activity remains) over the given period.

Dried formulations may be expected to have better physical and chemical stability than solutions, although would usually require reconstitution before use.

### Surfactant/Lipid Ratios

Insufficient quantities of surfactant may result in solutions with sub-optimal clarity, due to the presence of larger particles which disrupt the passage of light. Typically, the ratio of surfactant to lipid in the formulations of the present invention will be at least 0.5:1 on a weight basis (e.g. at least 0.75:1), especially at least 1:1, suitably at least 1.25:1, more suitably at least 1.5:1 (for example at least 2.0:1, such as about 2.5:1).

Excess quantities of surfactant may not provide substantial benefit (such as with respect to clarity or stability) and their use may therefore be unnecessarily wasteful and undesirable in pharmaceutical (or cosmetic) applications were large amounts of surfactant may be irritating. Suitably the ratio of surfactant to lipid in the formulations of the present invention will be 25:1 or lower on a weight basis, especially 15:1 or lower, in particular 12:1 or lower, such as 7:1 or lower (e.g. 5:1 or lower, 3.5:1 or lower or 3:1 or lower).

Suitably the surfactant to lipid ratio will be in the range 15:1 to 1:1, especially in the range 10:1 to 1.25:1, in particular 10:1 to 1.5:1 (e.g. 10:1 to 2:1).

The precise minimum ratio of surfactant to lipid which provides solutions of a desired clarity or stability level may vary to some degree between different surfactant/lipid combinations. Suitably, the ratio of surfactant to lipid will be sufficient to provide a solution of less than 150 FNU, especially less than 100 FNU, in particular less than 50 FNU (for example less than 25 FNU).

The presence of a co-surfactant and/or active agent (also the identity and the actual quantity thereof present) may also impact the ratio of surfactant to lipid necessary to obtain a desired clarity/stability level.

### Co-Surfactant

The presence of a small quantity of co-surfactant material may enhance the ability of the main surfactant to solubilise lipid (in particular lipid mixtures). This co-surfactant can take the form of a low molecular weight material, such as lyso (i.e. monoacylated) phospholipids, including the naturally occurring lyso-phospatidyl choline (lyso-PC) which is available under the tradename S LPC from Lipoid GmbH. Alternatively, the co-surfactant may be in the form of a polymeric surfactant material, such as the synthetic block copolymer polyoxyethylene/polyoxypropylene known as a poloxamer and supplied by BASF Corporation (e.g. the specific grade known under the tradename Lutrol® F127). The co-surfactant may also be a combination of more than one surfactant. The co-surfactant will typically have a high HLB (e.g. 18-20) relative to the main surfactant.

Suitably, co-surfactant is added in an amount equivalent to between 0.1-5% of the weight of lipid in the composition, especially 0.5-2.5% and in particular 0.75-1.5% (for example about 1%).

In one embodiment of the invention the co-surfactant is a block copolymer of polyoxyethylene/polyoxypropylene (for example having a molecular weight of 5000 to 15000 Da, in particular 10000 to 13000 Da, such as around 12700 Da as is found in Lutrol® F127). In a second embodiment of the invention the co-surfactant is lyso-PC.

It may be noted that certain lipid extracts may already contain lyso-PC, however, this does not preclude the addition of a co-surfactant (although high lyso-PC lipids may not benefit from the addition of co-surfactant to the same extent as low lyso-PC lipids).

Lyso-PC as co-surfactant may be added either in its pure form (e.g. S LPC from Lipoid GmbH), or as one component of a lipid mixture (e.g. a high lyso-PC content lecithin, such as those having at least 10% lyso-PC content by weight, especially at least 15% lyso-PC by weight). An exemplary high lyso-PC content lecithin is SL 80-3 from Lipoid GmbH. The addition of lyso-PC co-surfactant as a component of a high lyso-PC content lipid mixture is desirable due to the relatively high cost of the pure material.

### Physical Form

The formulations of the present invention may be in the form of an aqueous solution, especially a clear aqueous solution (e.g. a stable clear aqueous solution), suitably a clear and colourless aqueous solution (e.g. a stable clear and colourless aqueous solution). However, for ease of transportation and handling, once prepared, the compositions may be dried (e.g. by freeze-drying, or such like) to form a solid which has the benefits of being lower in both volume and weight.

In one embodiment the formulation is in the form of an aqueous solution. Aqueous solutions include aqueous semi-solids, such as gels. In a further embodiment the formulation is in dried form (for example as a powder, resin or flake). Suitably compositions of the invention in dried form can be reconstituted into aqueous solution to provide aqueous solutions.

Suitably an aqueous solution will contain at least 60% water by weight, such as at least 70%, especially at least 80%, in particular at least 90% (e.g. at least 95%, or at least 99%).

Suitably dried formulations will be substantially free of water, for example containing less than 5% water by weight, especially less than 2.5%, in particular less than 1.0%, such as less than 0.25%.

Aqueous solutions of may be prepared at relatively high concentrations, although high concentration aqueous formulations may demonstrate an increased viscosity. In one embodiment of the invention there is provided an aqueous solution comprising more than 0.001 and less than 10% by weight of the formulation of the invention, such as less than 5% or less than 2.5% (the percentage being determined by the dry weight of formulation of the invention relative to the total weight of composition with water). In a second embodiment of the invention there is provided an aqueous solution comprising 10-20% by weight of the formulation of the invention. In a third embodiment of the invention there is provided an aqueous solution comprising greater than 20% by weight of the formulation of the invention, such as up to 30% by weight.

BT type A is currently given by intra-muscular injection for example in the treatment of glabellar rhytids at a dose level of 20-100 Units (U). Consequently, treatment with a trans-dermal topical formulation may benefit from a solution containing 5-500 U/ml, such as 10-100 U/ml (e.g. 25-50 U/ml) of BT type A. Also of interest are formulations of the invention which are aqueous solutions containing 50-300 U/ml of BT. A typical dose of BT will depend on the area to be treated, though will usually be in the range of 5-500 U. Typically, 50-200 U are applied per 10 cm² area.

### BT

As used herein, the term BT refers to botulinum toxin or a derivative thereof. Thus, the BT to be used in the formulations of the invention may have the amino acid sequence of a natural BT polypeptide (whether natural origin or recombinant produced), or a fragment thereof (e.g. a fragment of at least 30% of the full length sequence, such as at least 50%, at least 75% or at least 90%) or may be an artificial construct. The BT will typically have a molecular mass in the region of 130-170 kDa and contain functional domains responsible for recognition, translocation and protease activity.

In one embodiment of the invention the BT will have the amino acid sequence of a natural BT polypeptide. The skilled person will recognise that a natural BT polypeptide need not be obtained by purification from Clostridium botulinum culture but could also be recombinantly produced in another cell type.

The natural BT peptide may be a type A sequence (e.g. subtype A1, subtype A2, subtype A3, subtype A4 or subtype A5). The natural BT peptide may be a type B sequence. The natural BT peptide may be a type C (e.g. subtype C1 or subtype C2) sequence. The natural BT peptide may be a BT type D sequence. The natural BT peptide may be a BT type E sequence. The natural BT peptide may be a BT type F sequence. The natural BT peptide may be a BT type G sequence.

In respect of artificial BT constructs, they will typically contain functional domains responsible for recognition, translocation and proteolytic activity which may be obtained from natural BT sequences (e.g. one domain may be taken from a first natural BT, a second domain may be taken from the first or a second natural BT and a third domain may be taken from the first, second or a third natural BT). Artificial BT constructs will suitably cleave SNAP-25, vesicle-associated membrane protein and/or syntaxin. Artificial BT constructs may, for example, contain functional domains responsible for translocation and proteolytic activity which are obtained from natural BT sequences (the same or different natural BT sequences) and a recognition domain which is not derived from a natural BT and is, for example, a polypeptide adapted to recognise a target cell which is not naturally targeted by BT (such as a non-neuronal cell). Artificial BT constructs will suitably target synaptic vesicle protein 2 or synaptotagmin. The relative ordering of functional domains within an artificial BT construct may be changed, such that the recognition domain is located in the N-terminal portion of the HC and the translocation domain in the C-terminal portion.

Derivatives include fragments of the aforementioned proteins, and proteins comprising fragments of the aforementioned proteins. Example fragments include functional domains, such as LC, HC C-terminus and HC N-terminus.

Suitably the BT will comprise (e.g. consist of) a sequence having at least 75% identity to polypeptide of SEQ ID Nos: 1-11, such as at least 90% identity, in particular at least 95% identity, at least 98% identity, at least 99% identity or even 100% identity.

Percent amino acid sequence identity is defined as the percentage of amino acid residues in a candidate sequence that are identical with the amino acid residues in the reference sequence, after aligning the sequences and introducing gaps, if necessary, to achieve the maximum correspondence. Alignment for purposes of determining percent amino acid sequence identity can be achieved in various ways that are within the skill in the art, e.g. manually, or by using publicly available computer software such as BLAST, BLAST-2, ALIGN, ALIGN-2 or Megalign (DNASTAR) software. The % amino acid sequence identity of a candidate amino acid sequence to reference amino acid sequence is the percentage of amino acid residues which are found to be identical relative to the total number of amino acid residues in the reference sequence.

BLAST and BLAST 2.0 algorithms, which are described in Altschul et al., Nuc. Acids Res. 25:3389-3402 (1977) and Altschul et al., J. Mol. Biol. 215:403-410 (1990), respectively, may be used to determine sequence identity. Software for performing BLAST analyses is publicly available through the National Center for Biotechnology Information (website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

Suitably, when used in the present invention the BT is not complexed to other proteins. If complexed to other proteins it may be necessary to remove these before preparation of the formulations of the invention.

### Manufacture

Methods for the production of macromolecular assemblies of use in the present invention are described in detail within WO99/009955, WO2006/129127 and WO2008/065451. These applications demonstrate the ability to use a broad range of surfactants and lipid types for the creation of stable macromolecular assemblies.

When incorporating temperature sensitive agents into macromolecular assemblies, suitably manufacture will be undertaken under conditions which do not result in a significant loss of BT activity. BT is susceptible to loss of activity at elevated temperatures; therefore it is desirable to prepare macromolecular assemblies at as low a temperature as possible.

Various approaches are available to reduce the required processing temperature, for example, the use of a lipid which has a relatively low phase transition temperature. Alternatively, a mixture of lipids may be used, which mixture has a low phase transition temperature. Certain surfactants may be better suited to low temperature processing than others (e.g. polysorbate 20). Knowles T et al Journal of the American Chemical Society 2009 131(22):7484-7485 describes the incorporation of a number of proteins into macromolecular assemblies. By low phase transition temperature is suitably meant a phase transition temperature below about 25° C. An example of a low phase transition temperature lipid is 1,2-dioleoyl-sn-glycero-3-phosphocholine (DOPC).

Formulations of the present invention may suitably be prepared by mixing an aqueous solution of a surfactant with an aqueous emulsion containing lipid and BT. Optionally, mixing will be performed at a modestly elevated temperature (e.g. up to 50° C., desirably up to 40° C., in particular up to 30° C.). BT can also be sensitive to excessive agitation, therefore mixing should ideally be gentle.

The surfactant solution may be prepared by dissolving the surfactant in water, optionally with stirring and heating (for example up to approximately 50° C., desirably up to 40° C., in particular up to 30° C.). The lipid emulsion may be prepared by mixing dried lipid with water, suitably with stirring and heating (suitably to a temperature above the phase transition temperature of the lipid component, for example up to approximately 50° C., desirably up to 40° C., in particular up to 30° C.), followed by homogenisation. BT may then be added to the lipid emulsion. Suitably the surfactant solution and lipid/BT emulsion are mixed by the addition (e.g. the slow addition) of lipid emulsion to the surfactant solution.

In an alternative embodiment of this process, surfactant need not be prepared in aqueous solution and instead may be added directly to the emulsion of lipid/BT. Suitably, addition will be performed under mixing, optionally at a modestly elevated temperature (e.g. up to 50° C., desirably up to 40° C., in particular up to 30° C.).

In a yet further embodiment, BT may be mixed with a preformed macromolecular assembly. Should the properties of the surfactant be pH dependent, the pH of solutions may be adjusted using acids or bases as appropriate. Compositions for use in the fields of cosmetics or pharmaceuticals will typically utilise acids and/or bases which are physiologically acceptable. Physiologically acceptable acids include hydrochloric acid. Physiologically acceptable bases include sodium or potassium hydroxide.

Co-surfactant, in particular when present as a component of a high lyso-PC lipid extract, will typically be mixed to form a fine aqueous emulsion prior to the addition of the lipid component. The resultant emulsion is then added to the aqueous surfactant solution. When added as a pure co-surfactant it will typically be combined with the surfactant prior to the formation of the aqueous solution thereof.

In a further aspect of the present invention there is provided a method for the production of a formulation comprising BT, lipid and surfactant wherein the surfactant and lipid are in the form of macromolecular assemblies, comprising the steps of:
(i) Preparing an aqueous solution of surfactant;
(ii) Preparing an aqueous emulsion of lipid and BT; and
(iii) Mixing the aqueous lipid/BT emulsion and aqueous solution of surfactant; such that macromolecular assemblies are formed.

Optionally, the aqueous emulsion of lipid and BT may be prepared by:
(a) Creation of an emulsion of liposomes with internal aqueous phases of pH 3-5, such as about pH 4
(b) Extraction of acidic liposomes and removal external buffer solution
(c) If necessary, disassociate carrier protein haemagglutinin from BT by adjustment of a solution of BT to pH to 7.5-9, such as about pH 8.5
(d) Adding disassociated BT solution at pH 7.5-9 to acidic liposomes.

Optionally, co-surfactant is included in the aqueous solution of (i) or the aqueous emulsion (ii).

If desirable, a further optional step of removing the water may be performed to provide dried formulations of the present invention.

Formulations of the present invention in the form of an aqueous solution may be dried (e.g. by freeze-drying) to produce compositions of the present invention in dry form. Dried formulations of the invention may be readily reconstituted into aqueous solution by the addition of water with stirring and suitably with warming.

The present inventors have surprisingly discovered that maltodextrin can be used to assist formation macromolecular assemblies at lower temperature, which may be of use in circumstances where a heat labile agent is incorporated into macromolecular assemblies (e.g. proteins) or where a reduced energy input may be desirable. To minimise the possibility of Maillard reactions, the maltodextrin will ideally have a low dextrin equivalent value (DE), such as less than 10.

Accordingly, there is provided a process for the manufacture of a composition comprising a lipid and a surfactant, wherein the lipid and surfactant are in the form of macromolecular assemblies comprising the steps of:
(i) Preparing an aqueous solution of surfactant;
(ii) Preparing an aqueous emulsion of maltodextrin and lipid; and
(iii) Mixing the aqueous maltodextrin and lipid emulsion and aqueous solution of surfactant;
such that macromolecular assemblies are formed.

The aqueous emulsion of maltodextrin and lipid is generally prepared by adding lipid to a solution of maltodextrin. Optionally, co-surfactant is included in the aqueous solution of (i) or the aqueous emulsion (ii). If desirable, a further optional step of removing the water may be performed to provide dried compositions. Incorporation of active agents (e.g. BT) will normally be facilitated by mixing with the aqueous emulsion of maltodextrin and lipid prior to step (iii). In this low temperature process, the preferred surfactants, lipids and other properties are the same as those described previously in relation to BT formulations of the invention and recited in the claims.

In an alternative embodiment of this process, surfactant need not be prepared in aqueous solution may be added directly to the emulsion of maltodextrin and lipid. Suitably, addition will be performed under mixing, optionally at a modestly elevated temperature (e.g. up to 50° C., desirably up to 40° C., in particular up to 30° C.).

There is also provided the use of maltodextrin to enable the formulation of macromolecular assemblies of lipid and surfactant at a temperature below that at which they would otherwise form.

Where utilised, maltodextrin will typical be present at a lipid to maltodextrin ratio of at least 10:1, suitably at least 5:1. Excessive amounts of maltodextrin are not generally beneficial, such that the lipid to maltodextrin ratio will typically be less than 1:10, such as 1:5. The lipid to maltodextrin ratio is suitably between 5:1 and 1:5, such as 2:1 and 1:3.

### Preparations

In general a formulation of the present invention will be incorporated into a cosmetic or pharmaceutical preparation which is tailored to suit a particular purpose, manner of use and mode of administration.

Formulations may be mixed with one or more cosmetic or pharmaceutically acceptable carriers or excipients (anti-oxidants, preservatives, viscosity modifiers, colourants, flavourants, perfumes, buffers, acidity regulators, chelating agents, or other excipients), and optionally with other therapeutic ingredients if desired. Such preparations may be prepared by any of the methods known in the art, and may for example be designed for topical or parenteral (including intravenous, intra-articular, intra-muscular, intra-dermal and subcutaneous) administration. Administration by oral or inhalation routes are theoretically possible, though less likely to be used in practice.

Pharmaceutical preparations are made using pharmaceutically acceptable components, especially biodegradable components. Some of the phospholipids described in this application are used for parenteral nutrition and are therefore likely to be broken down fairly readily in the body without causing serious problems. A number of the surfactants described herein are available in pharmaceutical grades. Preparations for parenteral delivery will suitably be sterile.

Formulations of the present invention are believed to be particularly suitable for facilitating the topical delivery of BT (e.g. topically for local effect, or alternatively topically for systemic effect), in particular topical delivery to a mammal (e.g. a human). Topical delivery may, for example, be via a mucosal surface. Topical delivery will typically be via the dermal surface. Formulations of the present invention are believed to be particularly suitable for the delivery of BT to (or through) the skin, in particular to (or through) the skin of humans.

When delivering active agents to the skin it is generally important that the particle size be less than that of the lipid interstices found between the corneocytes within the outer layer of the skin, in order for the material to be adequately absorbed into the stratum corneum. The inter-corneocyte interstices have relatively small thickness, hence, particles should desirably be sized to be absorbed efficiently. The macromolecular assemblies described in this application are well suited to penetrating the inter-corneocyte lipid layer and could therefore be used to deliver materials such as BT.

Although formulations for repeated application to the skin may be slightly acidic, typically being in the pH 5.0-7.5 range, particularly pH 5.5-7.5, formulations for application to other sites, or for internal administration, should typically be maintained around pH 6.5-7.5. Formulations specifically for application to the eye are ideally in the range pH 7.1-7.8, more particularly pH 7.3-7.6 (Carney, L G and Hill, R M Arch. Ophthalmol. 1976 94(5):821-824).

Preparations for topical application may include, for example, anti-oxidants (e.g. alpha-tocopherol, butylated hydroxyanisole (BHA) or butylated hydroxytoluene (BHT)), preservatives (e.g. 2-phenoxyethanol, sorbic acid or parabens), viscosity modifiers (e.g. water soluble gums and resins, such as xanthan gum, carboxymethyl cellulose or lightly cross-linked synthetic polymers such as carbomers, e.g. Carbopols), colourants, flavourants, perfumes, buffers, acidity regulators, chelating agents (e.g. such as EDTA, sodium edetate, disodium edetate or calcium disodium edetate), penetration enhancers and anti-tack agents. Suitable carbomers include Carbopol® 980 and Ultrez® 20. Other suitable gelling agents include: carbomers—Carbopol® Ultrez 10, Carbopol® Ultrez 21, Carbopol® Aqua-SF1, Stabileze® QM; cellulose ethers—Natrasol® 250 and Blanose® 7HF. Other suitable preservatives include Nipaguard PDU (e.g. at around 0.5% by weight), Nipaguard DMDMH (e.g. at around 0.2% by weight), Germaben® II-E (e.g. at around 1% by weight), Suttocide® A (e.g. at around 0.5% by weight), Euxyl® K500 (e.g. at around 1.5% by weight), Euxyl® PE9010 (e.g. at around 1% by weight) and Euxyl® SC50 (e.g. at around 0.2% by weight).

Preparations for topical application may be incorporated into hydrogel patches (i.e. 3-dimensional gels of fixed structure, such as those available from Telic S.A. (Spain)). Other biocompatible hydrogel patches are those supplied by Allmi-Care Limited (Nottingham, UK). Application utilising hydrogels may be advantageous in that: (i) the hydrogel patch may act as a convenient repository for prolonged administration and/or (ii) the hydrogel patch may provide a quantifiable dosage form, such that the quantity of active agent administered can be effectively controlled. Additionally, hydrogel patches may aid absorption by ensuring that skin is fully hydrated.

Delivery of active agents using hydrogel patches may be enhanced by the use of electrical stimulation techniques, such as transcutaneous electrical nerve stimulation (TENS). An alternative electrical stimulation technique is Interferential TENS.

Thus, there is provided a cosmetic preparation comprising a formulation of the invention and a cosmetically acceptable carrier or excipient.

Cosmetic uses of the formulations of the invention are those where muscle is relaxed to achieve an aesthetic effect—e.g. the treatment of facial lines, creases and wrinkles or the treatment of the depressor anguli oris, nasolabial folds, mentalis, medial and lateral brow lifts, to lessen shadows and maintain a smooth appearance. An alternative use of the formulations of the invention is for reduction of sweating in hyperhydrosis. A further use of the formulations of the invention is for reduction of sweating for aesthetic considerations.

There is also provided a pharmaceutical preparation comprising a formulation of the invention and a pharmaceutically acceptable carrier or excipient.

Pharmaceutical uses include the treatment of: disorders associated with smooth muscle or gland activity including hyperhydrosis; pain, such as headaches; disorders associated with skeletal muscle including tremors, spasms and dystonias.

Accordingly, there is also provided a formulation of the present invention for use in therapy.

Formulations, cosmetic preparations and pharmaceutical preparations according to the present invention may comprise additional therapeutic agents or be utilised in conjunction with additional therapeutic agents (e.g. in combined or separate formulations, to be administered through the same or different routes, at the same, sequentially or different times), The identity of the additional therapeutic agent will be dependent upon the disorder to be treated, prevented or ameliorated by the administration of BT. The additional therapeutic agent may be intended to improve the effect of the BT, address aspects of a disorder not satisfactorily dealt with by BT alone or reduce/treat side-effects associated with BT administration.

Administration according to the present invention, for example topically, may result in a more rapid onset of effect than administration of conventional BT formulations through the conventional injection route.

The invention therefore provides a method for the treatment, prevention or amelioration of:
(i) a disorder associated with smooth muscle or gland activity including hyperhydrosis;
(ii) pain, such as headaches; disorders associated with skeletal muscle including tremors, spasms and dystonias; or
(iii) a disorder associated with skeletal muscle including tremors, spasms and dystonias; comprising the administration of a formulation of the present invention to a subject in need thereof. Suitably the subject will be a human.

The invention also provides a method for the treatment, prevention or amelioration of facial lines, creases and wrinkles comprising the administration of a formulation of the present invention to a subject in need thereof. Suitably the subject will be a human.

After topical administration of a formulation of the invention to an area it may assist absorption by hydrating the skin, such as by occlusion with a barrier which is impermeable to water vapour, such as a plastic membrane such as PVC or the like.

### Miscellaneous

Suitably the aqueous compositions of the invention do not comprise an oil in water or water in oil emulsion.

Suitably the surfactant is not an ethoxylated PPG acyl ether. Suitably the surfactant is not an ethoxylated PPG ether. Suitably the surfactant is not a propoxylated POE ether. Suitably the surfactant is not an ethoxylated glyceride. Suitably the surfactant is not a polyglycerol ester. Suitably the surfactant is not an acylated sorbitan ester. Suitably the surfactant is not a PEG non-sorbitan sugar ester. Suitably the surfactant is not a synthetic phospholipid. Suitably the surfactant is not a fatty acid. Suitably the surfactant is not an ester of an alpha-hydroxycarboxylic acid. Suitably the surfactant is not an anionic phosphate based surfactant.

Suitably the surfactant is not cocamidopropyl betaine. Suitably the surfactant is not sodium cholate, sodium deoxycholate, sodium laureth sulphate or sodium lauryl sulphate.

The following Examples are non-limiting and are provided to illustrate the preparation and use of compositions according to the present invention such that a person skilled in the art may more readily appreciate the nature of the invention and put the invention into practical effect.

### EXAMPLES OF THE INVENTION

### Example 1

### The Use of Maltodextrin to Aid Low Temperature Formation of Macromolecular Assemblies Using Ethoxyalkylated Aromatic Alcohol Ether Surfactants

A range of surfactants were tested against a standard maltodextrin/lipid emulsion for their suitability to be used in the present invention, as indicated by their ability to solubilise a maltodextrin/lipid mixture through the formation of macromolecular complexes.

### Method

Each surfactant was tested using a standard maltodextrin/lipid emulsion containing 2% C* Dry MD™ 01958 (maltodextrin) and 1% S-75 (lipid). A stock emulsion was prepared of 2% C* Dry MD™ 01958 and 1% S-75.2% C* Dry MD™ 01958 was dissolved into water at room temperature whilst stirring, with the conditions maintained for a further 5 minutes. 1% S-75 was then added, followed by continued stirring at room temperature for 30 minutes until a uniform emulsion was present.

Each surfactant was then added at the required volume (7% w/w final concentration) to an aliquot of the C* Dry MD™ 01958/S-75 emulsion whilst stirring at room temperature, with the conditions maintained for a further 30 minutes.

For a quantitative analysis of the clarity of the aqueous solutions of surfactant and maltodextrin/lipid, samples were examined using a turbidity meter (Nephla, from Hach-Lange). The turbidity meter was calibrated prior to use, with two known standards (0 and 40FNU).

### Surfactants

The HLB values in the Tables below are based on a combination of the values reported by the manufacturer for the commercial product and those given in the literature (e.g. McCutcheon's Volume 1: Emulsifiers & Detergents, International Edition, MC Publishing Company, Glen Rock, N.J., USA, 2005; Handbook of Industrial Surfactants, M Ash & I Ash, Gower Publishing Company, Aldershot, England, 1993). An approximate average of reported values is given.

Surfactants utilised in this experiment are:
Surfac OP 5 (Octoxynol-5) supplied by Surfachem Group Ltd. (UK) CAS: 9002-93-1
Surfac OP 30 (Octoxynol-30) supplied by Surfachem Group Ltd. (UK) CAS: 9002-93-1
Igepal CA-720 (Octoxynol-12) supplied by Sigma-Aldrich Ltd. (UK) CAS: 9002-93-1
Igepal CO-890 (Octoxynol-40) supplied by Sigma-Aldrich Ltd. (UK) CAS: 9002-93-1
Sympatens-NP/090 (Nonoxynol-9) supplied by Kolb Distribution Ltd. (Switzerland) CAS: 9016-45-9
Tergitol® NP-10 (Nonoxynol-10) supplied by Sigma-Aldrich Ltd. (UK) CAS: 127087-87-0
Triton X-100 (Octoxynol-9) supplied by Sigma-Aldrich Ltd. (UK) CAS: 9002-93-1
Triton X-165 (Octoxynol-16) supplied by Sigma-Aldrich Ltd. (UK) CAS: 9002-93-1
Triton X-405 (Octoxynol-40) supplied by Sigma-Aldrich Ltd. (UK) CAS: 9002-93-1

### Lipids

S 75 is a purified soy extract containing 68-73% phosphatidyl choline. It is available from Lipoid GmbH.

### Maltodextrin

C* Dry MD™ 01958 supplied by Cargill Haubourdin SAS (France). D.E. Lane Eynon value of 7.5-9.9. CAS: 9050-36-6.

### Results

| | | | | Turbidity |
|---|---|---|---|---|
| Surf. | Tradename | Supplier | HLB | (FNU) |
| Octoxynol-5 | Surfac OP 5 | Surfachem | 10.4 | >150 |
| Nonoxynol-9 | Sympatens NP/090 | Kolb | 12.9 | 8.87 |
| Octoxynol-9 | Triton X-100 | Sigma | 13.5 | 7.75 |
| Nonoxynol-10 | Tergitol NP-10 | Sigma | 13.6 | 8.29 |
| Octoxynol-12 | Igepal CA-720 | Sigma | 14.5 | 4.32 |
| Octoxynol-16 | Triton X-165 | Sigma | 15.8 | 7.55 |
| Octoxynol-30 | Surfac OP 30 | Surfachem | 17.1 | >150 |
| Nonoxynol-40 | Igepal CO-890 | Sigma | 17.8 | >150 |
| Octoxynol-40 | Triton X-405 | Sigma | 17.9 | >150 |

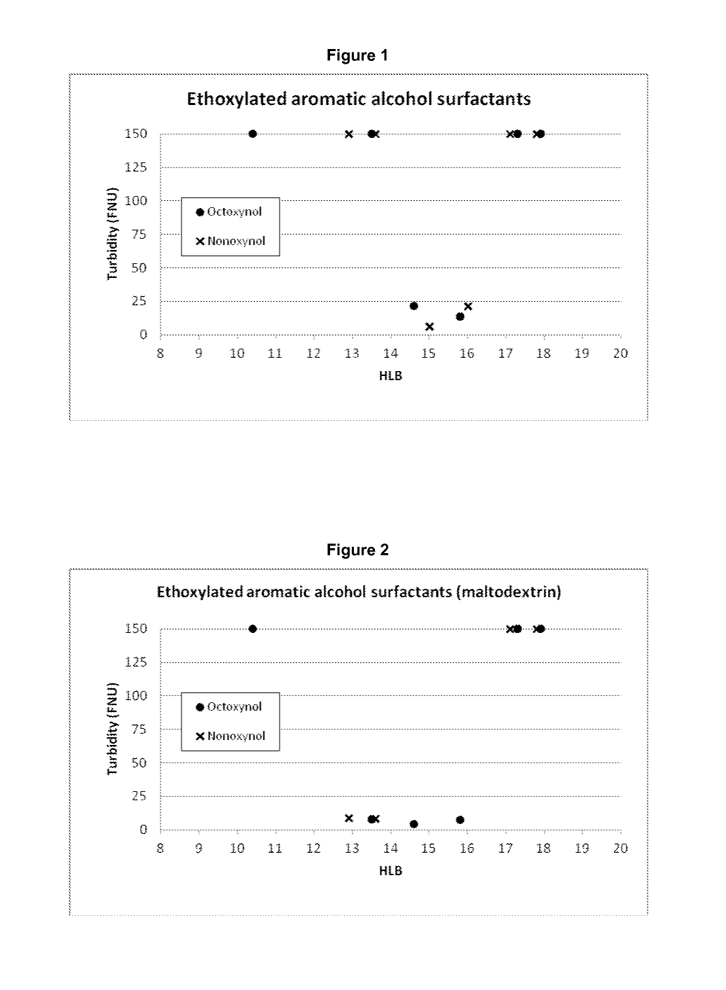

FIG. 2 illustrates these results, which may be compared to FIG. 1 (taken from WO2008/065451, where a maltodextrin free process was used at elevated temperature). It is clear that the presence of maltodextrin does not detrimentally impact the formation of the macromolecular assemblies. Indeed, the presence of maltodextrin seems to broaden the HLB range of surfactants within this class which are capable of forming macromolecular assemblies, despite the lower temperature used.

### Example 2

### The Use of Maltodextrin to Assist Low Temperature Formation of Macromolecular Assemblies Using a Range of Surfactants

A range of aqueous solutions of compositions of the invention containing surfactant and lipid (S-75) were prepared, with or without maltodextrin (C* Dry MD™ 01958/S-75). Brij 35P and Protasorb L-20 samples were prepared analogously to the previously described method in Example 1.

The SMA3000 samples were prepared as follows. A stock emulsion of lipid (with or without maltodextrin) was prepared at double the desired final concentration, but otherwise as described in Example 1. A stock solution of copolymer was prepared at double the desired final concentration by mixing of the hydrolysed polymer with the appropriate volume of water. Copolymer/lipid mixtures were then prepared by the dropwise addition of the lipid emulsion to an equal volume of polymer solution while stirring.

The pH of the resulting mixtures were lowered to approximately pH 6.5 before subsequently raising the pH to 6.8.

### Surfactant

Brij 35P (Laureth-23) supplied by Uniqema/ICI (Imperial Chemical Industries PLC) CAS: 9002-92-0.

Protasorb L-20 (Polysorbate-20) supplied by Protameen Chemicals Inc. (USA) CAS: 68154-33-6.

SMA3000 HNa was obtained from Sartomer Inc., it is a sodium salt form of hydrolysed SMA3000 (i.e. a styrene/maleic acid sodium salt) and contains a 3:1 ratio of styrene to maleic acid monomer units (i.e. is a blocky polymer). The polymer is supplied as a resin.

### Lipid

S-75 was as described in Example 1.

### Maltodextrin

C* Dry MD™ 01958 was as described in Example 1.

### Results

| | Clarity without | Clarity with |
|---|---|---|
| Surfactant | maltodextrin | maltodextrin (2%) |
| Brij 35P (7%) | 184 FNU | 128.1 FNU |
| Polysorbate 20 (7%) | 79.5 FNU | 59.5 FNU |
| SMA 3000 pH 6.8 (2.5%) | 101.4 FNU | 51.9 FNU |

As can be seen from the table above, maltodextrin aids the formation of macromolecular assemblies at lower temperatures, helping to ensure a high level of solution clarity (and by implication uniformly small particles) while ensuring that high sensitive components can still be incorporated.

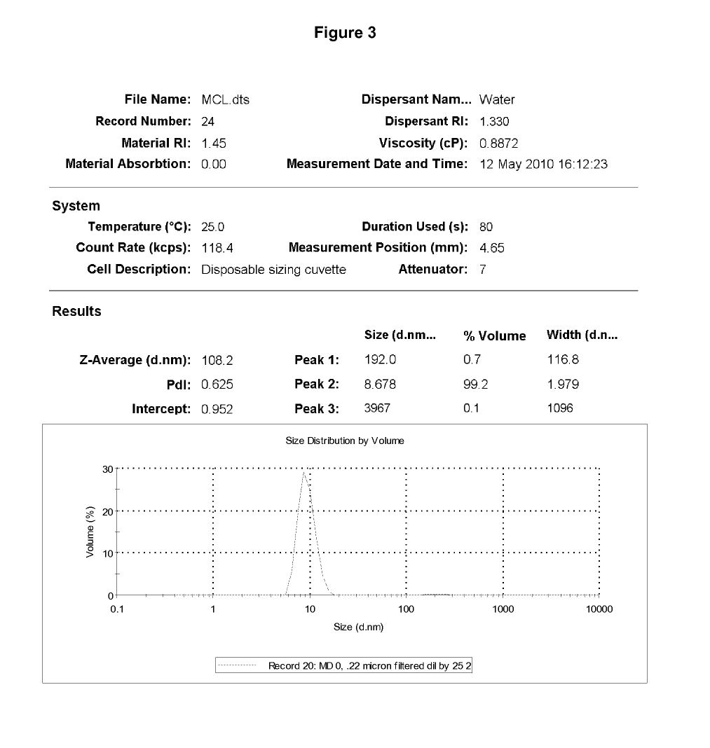

FIG. 3 provides a particle size analysis for a Brij35P sample with maltodextrin at 25:2 dilution. The dominant particle size being 8.68 nm and the polydispersity being 0.625. The preparation therefore contains uniformly small particles.

### Example 3

### Formulation of Botulinum Neurotoxin Type a Using Macromolecular Assemblies

Botulinum Neurotoxin Type A was formulated with macromolecular assemblies to investigate to illustrate the potential application in the field of pharmaceuticals and cosmetics.

### Method

Samples with and without maltodextrin were prepared.

For the example with maltodextrin, a stock emulsion was prepared of 2% C* Dry MD™ 01958 and 1% S-75.2% C* Dry MD™ 01958 was dissolved into water at room temperature whilst stirring, with the conditions maintained for a further 5 minutes. 1% S-75 was then added, followed by continued stirring at room temperature for 30 minutes until a uniform emulsion was present. The emulsion was then sonicated for 5 minutes at room temperature (Grant Ultrasonic Bath XB2).

For the example without maltodextrin, An emulsion of 1% S-75 was prepared by continued stirring at room temperature for 30 minutes until a uniform emulsion was present. The emulsion was then sonicated for 5 minutes at room temperature (Grant Ultrasonic Bath XB2).

The lipid emulsions were then added to 100 unit Botulinum neurotoxin type A (Xeomin®) vial with continued stirring at room temperature for 5 minutes, to achieve a final concentration of 25 units/ml. pH of the emulsion was then adjusted with NaOH(aq), drop-wise addition, to pH-8.5, to induce disassociation of the toxin molecule from human serum albumin or residual non-toxin hemaglutinin protein and a non-toxin/non-toxic nonhemagglutinin proteins, stirring at room temperature is maintained throughout. After 5 minutes pH reduced with HCl(aq) drop-wise to pH˜7.0, stirring at room temperature was maintained throughout and continued for 5 minutes after pH adjustment. The resulting BT containing emulsions were then sonicated for 10 minutes at room temperature, before being stirred at room temperature for a further 10 minutes. Polysorbate 20 was then added (equivalent 7% w/w), stirring was maintained throughout and continued for a further 30 minutes after addition.

Once the composition was prepared it was visually examined to determine whether the surfactant component had solubilised the BT containing emulsions in the aqueous media. The clarity of the mixture was categorised as being clear if there was no significant visible opacity to the naked eye.

### Surfactant

Protasorb L-20 (Polysorbate-20) was as described in Example 2.

### Lipid

S-75 was as described in Example 1.

### Maltodextrin

C* Dry MD™ 01958 was as described in Example 1.

### Botulinum Neurotoxin Type A

Xeomin® (Botulinum Neurotoxin Type A) supplied by Merz Pharmaceuticals GmbH (Germany).

### Example 4

### An Aqueous Gel Preparation of Botulinum Neurotoxin Type A

The aqueous maltodextrin containing product of Example 3 was preserved by dissolving 1% Euxyl K500 into the medium whilst stirring at room temperature. The viscosity of the preserved solution was then modified by the addition of 1.5% Natrasol® 250 HBXR and stirred at room temperature until a homogeneous gel was formed.

### Preservative

Euxyl® K500 (Diazolidinyl urea, Sodium benzoate and Potassium sorbate) supplied by Schülke & Mayr GmbH (Germany). CAS: 78491-02-0, 532-32-1, 24634-61-5.

### Viscosity Modifier

Natrasol® 250 HBXR (Hydroxyethylcellulose) supplied by Ashland Aqualon Functional Ingredients (USA) CAS: 9004-62-0.

### Example 5

### Topical Application of an Aqueous Gel Preparation of Botulinum Neurotoxin Type A to Human Subjects

Material produced under Example 4 was tested on a number of human subjects as described below.

Subject 1 (Medically Trained Individual with Previous Experience of Receiving BT by Infection):

### Method

Three application sites with approximate doses:
(i) Left frontalis region—0.5 ml gel containing 12.5 U BT
(ii) Glabella region—0.5 ml gel containing 12.5 U BT
(iii) Righthand orbicularis-oculi region 0.5 ml gel containing 12.5 U BT

Gel was applied by digital application and allowed to absorb without occlusion.

### Result

0-30 mins after application:
Subject reported site (i) slightly hot sensation with initial erythema, which moved from the treated into the adjacent untreated area with time.

30-60 mins after application:
Site (i) firstly a diminution of erythema and loss of hot sensation, weakness detected and extra effort required to activate the frontalis muscle, a typical feeling associated with botulinum toxin treatment. Slight ‘halo effect’ on the wrinkle lines of frontalis region very localised to the mid-papillary line, approx 3 cm above the orbital rim. Site (ii) frown lines feel weaker.
   Subject 2 (Lay Person with No Previous Experience of Receiving BT by Injection):

### Method

Two application sites with approximate doses:
(i) Right frontalis region—0.5 ml gel containing 12.5 U BT
(ii) Lefthand orbicularis-oculi region 0.5 ml gel containing 12.5 U BT

Gel was applied by digital application and allowed to absorb without occlusion.

### Result

0-30 mins after application:
Subject reported site (i) mild tightening sensation on the frontalis muscle

30-60 mins after application:
Site (i) Tightness around mid-frontalis region, static lines were softer laterally over the right frontalis region, dynamic lines were unaltered.

### Conclusions

Effects similar to those experienced with conventional injection of botulinium toxin were observed, at a relatively low dose. The onset of action seemed to be unusually rapid.

### Example 6

### Evaluation of Skin Penetration of the Macromolecular Complexes

Near infrared chemical imaging (NIRCI) was used to evaluate the penetration of the macromolecular complexes in-vivo via sequential removal of skin layers though stripping of the skin with adhesive tape.

### Method

1 ml of a solution of the invention containing surfactant (Polysorbate 20, 2.5%), lipid (90H, 1%), co-surfactant (SL 80-30, 0.05%) and a dye having poor water solubility (D&C Red 27, 0.25%) was dosed on a 25×25 mm cross-linked adhesive hydrogel patch before being allowed to absorb for 1 hr. Gels were contacted with forearm skin for 30 minutes after which the skin surface was then briefly washed.

After washing, layers of the stratum corneum were removed by firmly applying a 2 cm wide section of Scotch™ Pressure Sensitive adhesive tape to the test area, and subsequently removing the tape section from the skin. Application of tape sections was then repeated until no further dye penetration was noted at a total of 16 strips being applied and removed.

Each strip was then assessed by NIRCI, with each sample first being illuminated with broadband NIR light. After interaction with the sample, the resulting diffusely reflected light was collected with imaging optics. Wavelength selection was performed with a high resolution Liquid Crystal Tunable Filter (LCTF), and the resulting wavelength selected radiation (6 nm bandpass at 1600 nm) was focused onto a focal plane array with 320×256 pixels. The pieces of tape were placed on top of the 100% reflectance standard to permit a transflectance measurement whereby the light passed through the sample twice to increase the possible sample signal available. Chemometric multivariate analysis, was applied to calculate the contribution of each relevant component at every pixel over the images. The contribution or abundance of each component in a pixel was given by a score value.

### Surfactant

Protasorb L-20 was as described in Example 3.

### Lipid

Phospholipon® 90 H, available from Phospholipid GmbH (Germany), is a hydrogenated soy lecithin extract of at least 90% phosphatidylcholine content and is approved for pharmaceutical and cosmetic use. It is generally used as an emulsifier and is known to form liposomes.

### Dye

D&C Red No. 27 supplied by Sun Chemical Corporation (USA). CAS: 84473-86-9.

### Results

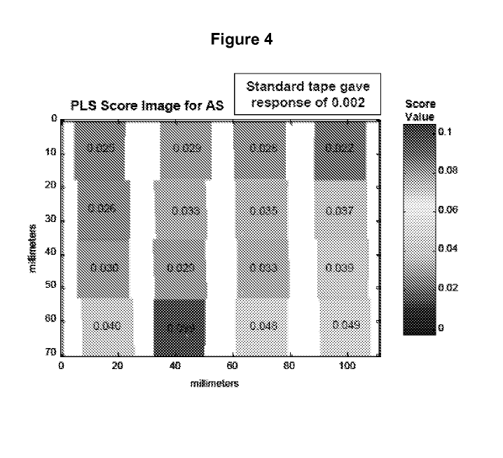

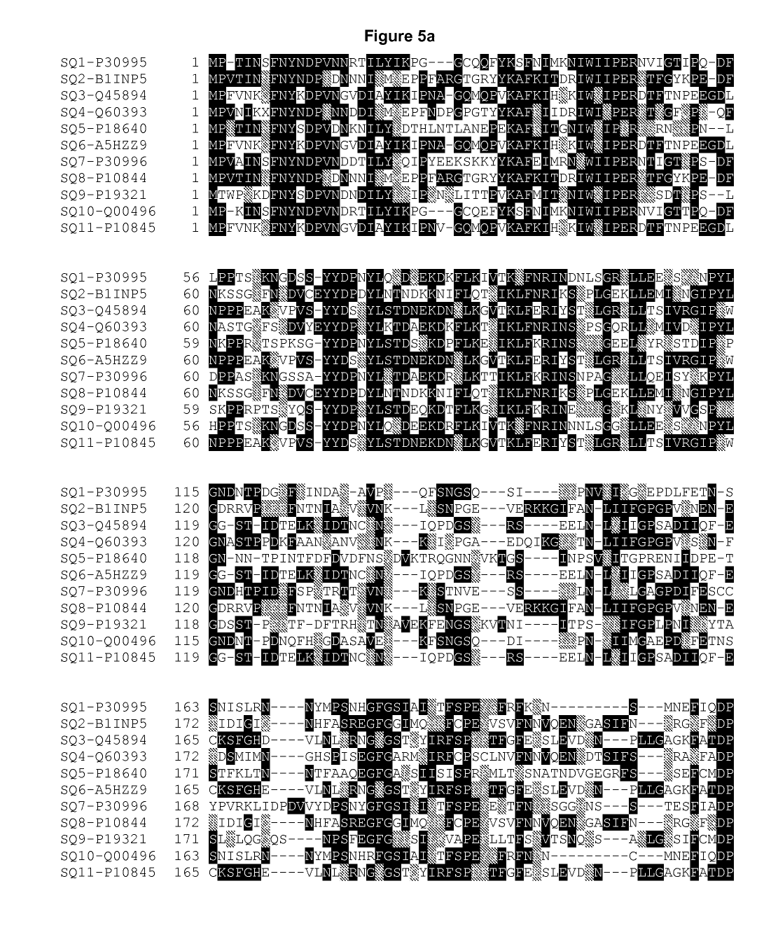

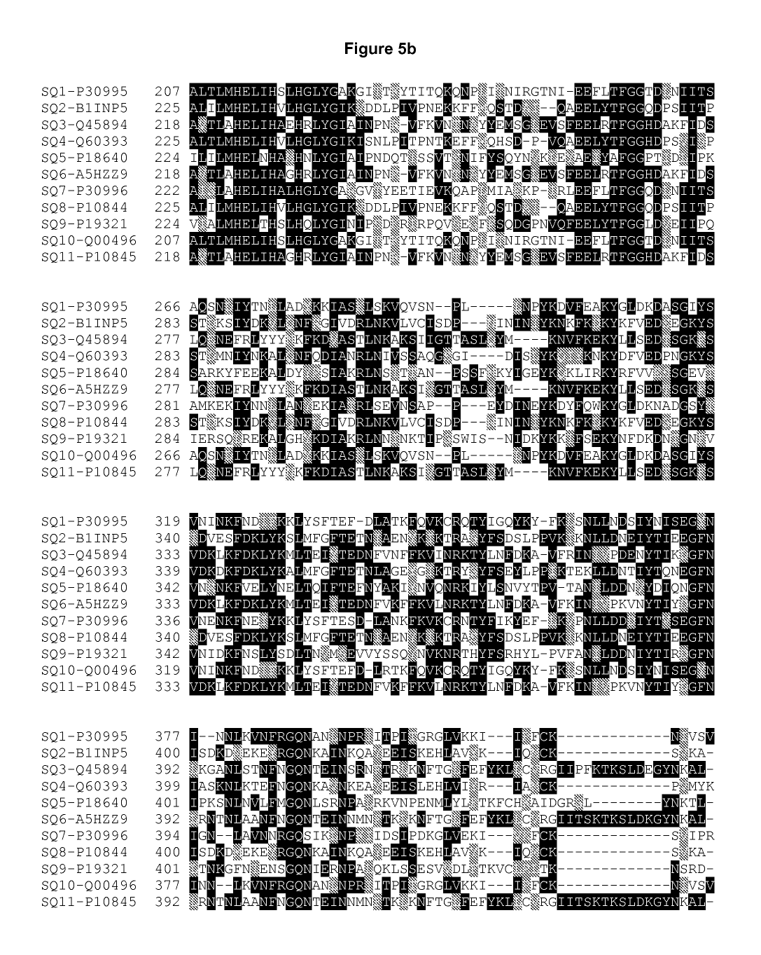

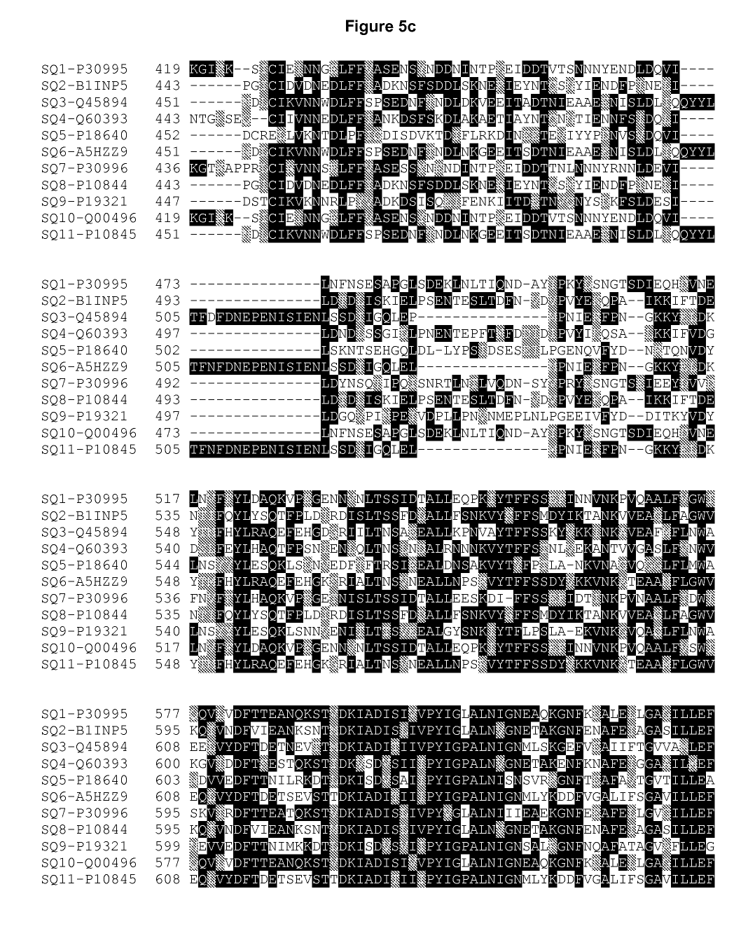

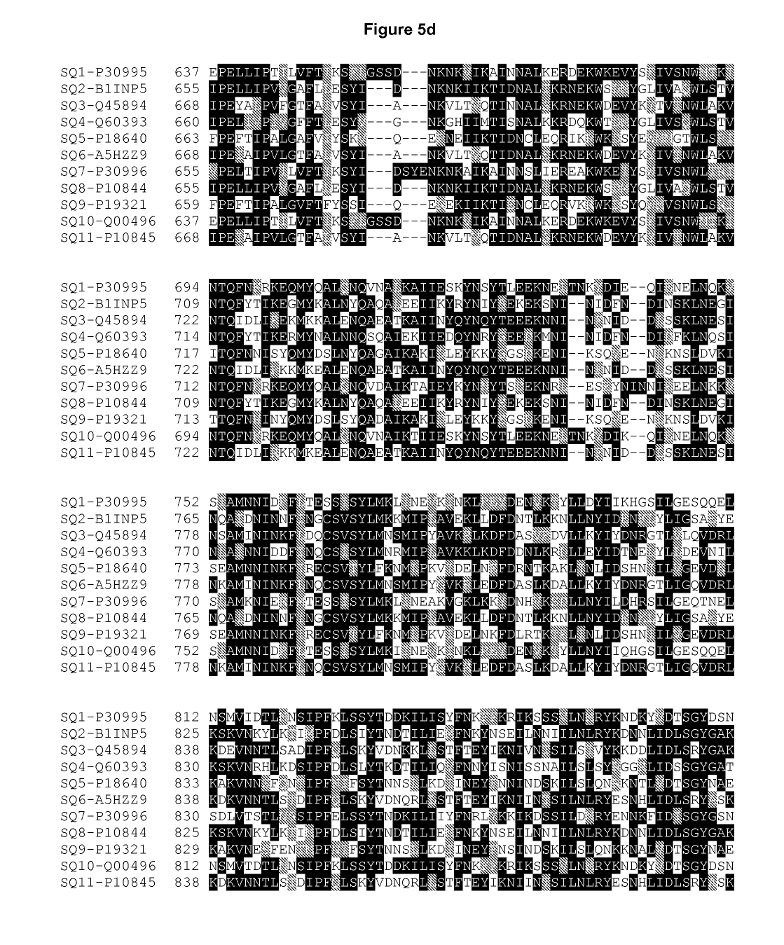

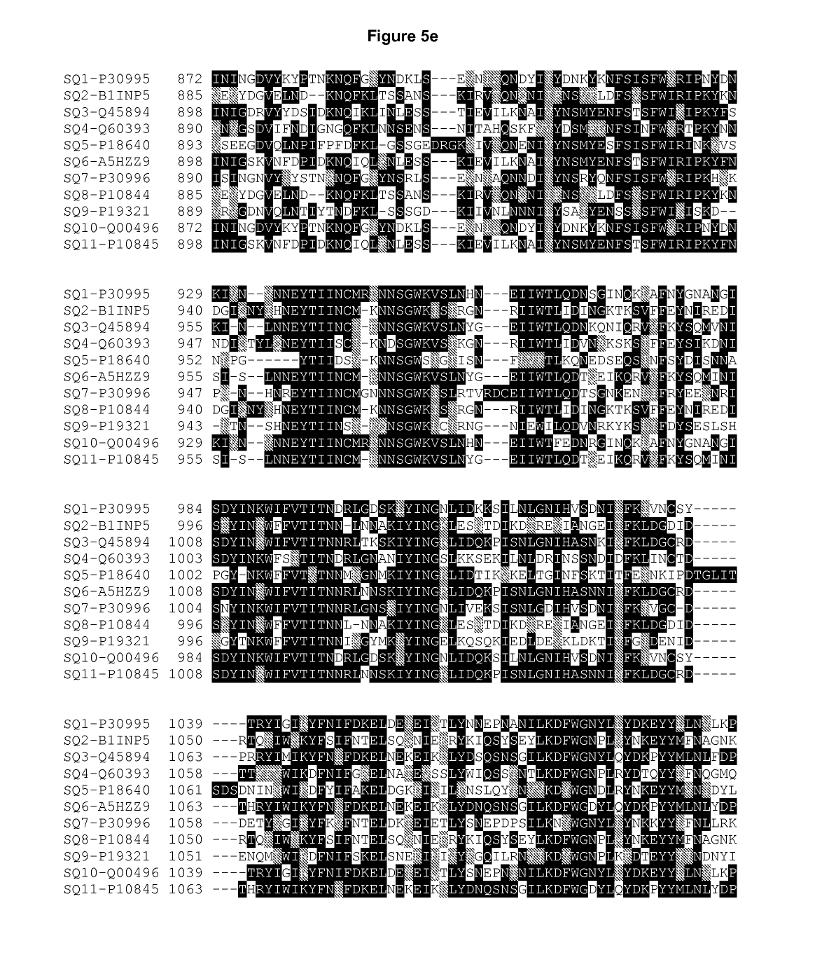

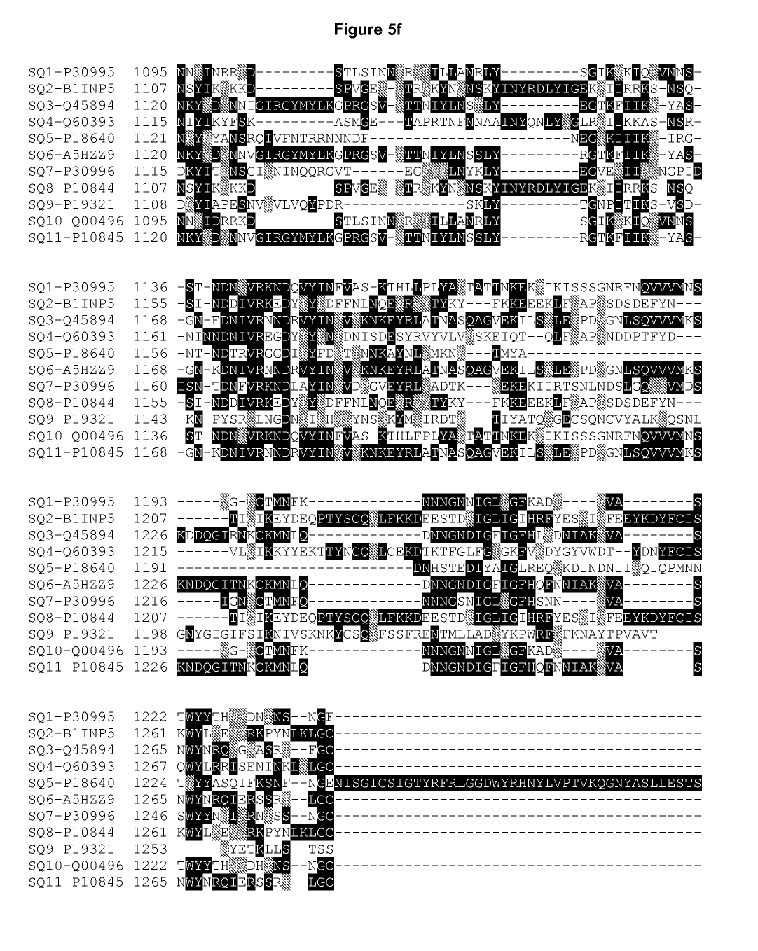

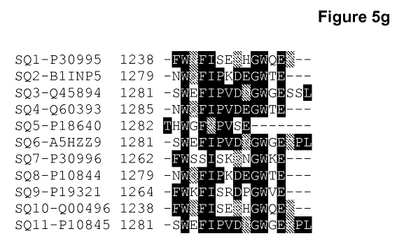

FIG. 4 provides an illustration of the results. Samples are ordered left to right, moving from top to bottom (i.e. the top row relates to layers 1-4, the second 2-8 etc.)

### Discussion

The score values demonstrate the greatest abundance of complexes within the 14^{th} strip. i.e. the 14^{th} strip to be removed from the skin. The macromolecular assemblies used in the present invention are therefore capable of penetrating the skin.

### Example 7

### Hydrosis Assessment

### Procedure for the Visual Evaluation of Hydrosis of the Forehead and Upper Lip

Iodine, in the form of Iodine tincture (2.5% w/v Iodine, 2.5% w/v potassium iodide, 89% v/v purified water and ethanol supplied by L.C.M. Ltd., Huddersfield, UK.) was applied sparingly with cotton wool to the foreheads of Subjects 1 and 2 and the upper lip of Subject 2. After 5 minutes cornflour was applied evenly over the test areas and the area immediately occluded using PVC film for 20 minutes.

During this period of time areas undergoing hydrosis or sweating will be highlighted by reaction between iodine and cornflour which only occurs in areas where moisture is present resulting in a blackening of the cornflour.

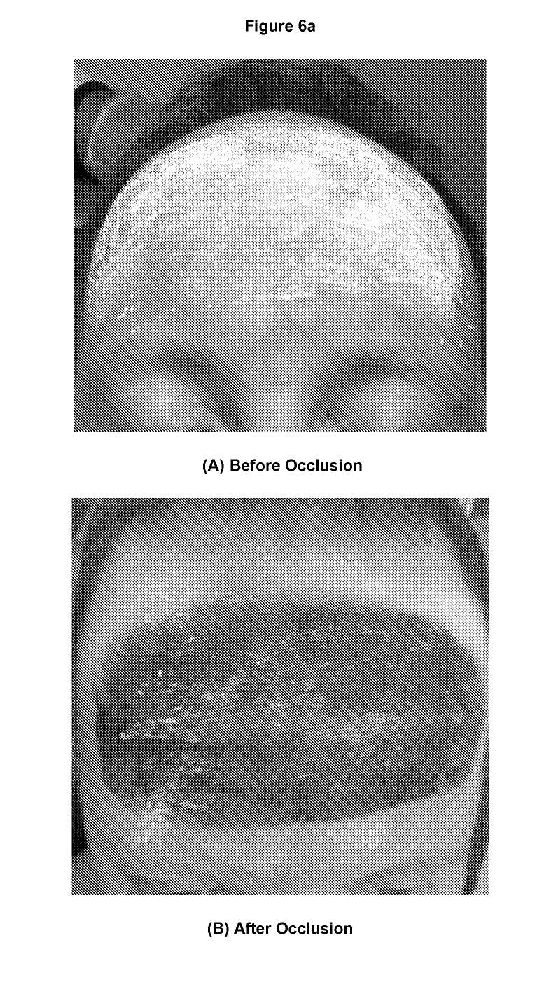

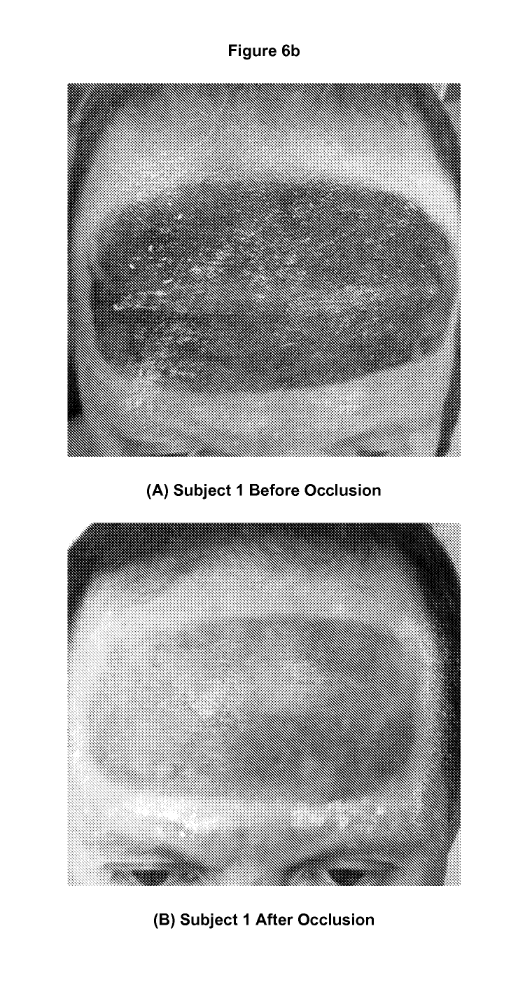

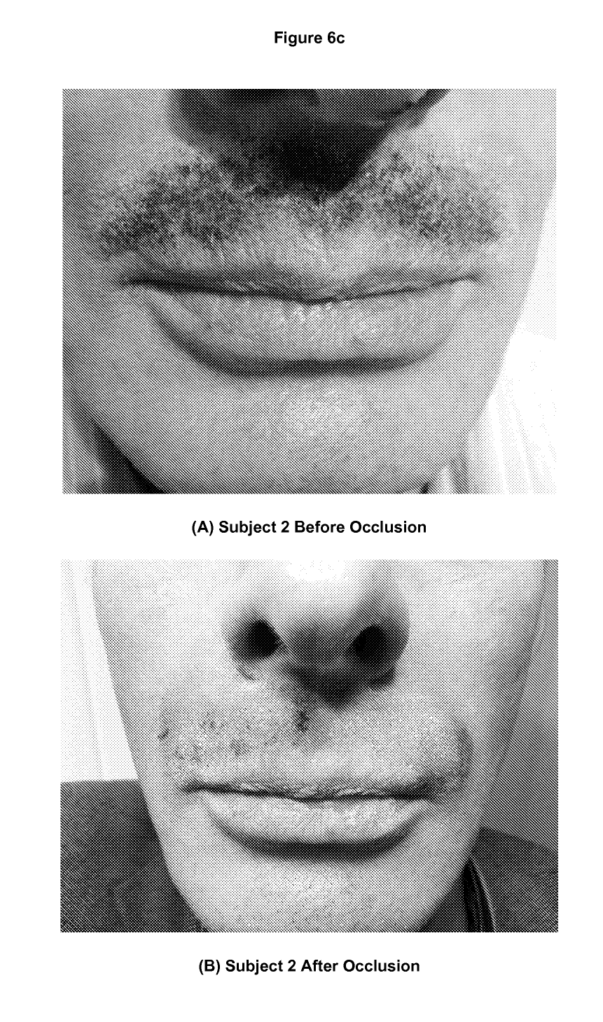

After 20 minutes and the removal of PVC film the area is photographed and visually assessed for evidence of hydrosis (See FIG. 6a, (A) Before Occlusion and (B) After Occlusion).

### Preparation of Formulation 7.1 (Formulated by Post Addition of BT to a Gel Containing Macromolecular Assemblies)

An emulsion of 1% Vav S-70 was prepared by continual stirring at room temperature for 30 minutes until a uniform emulsion was present. The emulsion was then sonicated for 5 minutes at room temperature (Grant Ultrasonic Bath XB2).

Polysorbate 20 was then added (equivalent 7% w/w), stirring was maintained throughout and continued for a further 30 minutes after addition.

Once the composition was prepared it was visually examined to determine whether the surfactant component had solubilised the emulsion in the aqueous media. The clarity of the mixture was categorised as being clear if there was no significant visible opacity to the naked eye.

The viscosity of the solution was then modified by the addition of 1.5% Natrasol® 250 HBXR and stirred at room temperature until a homogeneous gel was formed.

Prior to topical application to Subject 1 0.25 ml saline solution is added to 100 units of BT (Botox® Allergan Inc., USA) to solubilise the toxin. The BT solution was then added directly to 0.25 ml of the macromolecular assembly containing gel (mixing occurred as the BT solution and gel was drawn into a syringe).

0.5 ml of BT (100 units) solution/macromolecular assembly gel was applied to the right hand side of the forehead of Subject 1 (left side of photographs in FIG. 6b) and immediately occluded under PVC film for 30 minutes duration.

### Surfactant

Protasorb L-20 (Polysorbate-20) was used as described in Example 2.

### Lipid

Vav S-70 is a purified soy extract containing approximately 70% phosphatidyl choline. It is available from VAV Life Sciences Pvt. Ltd., India.

### Viscosity Modifier

Natrasol® 250 HBXR (Hydroxyethylcellulose) supplied by Ashland Aqualon Functional Ingredients (USA) CAS: 9004-62-0.

### Botulinum Neurotoxin Type A

BOTOX® (Botulinum Neurotoxin Type A) supplied by Allergan Inc., USA.

### Preparation of Formulation 7.2 (Formulated by pH Gradient Liposome Procedure)

An emulsion of 1% Vav S-70 was prepared by continual stirring at approximately 50° C. for 30 minutes in a solution buffered to pH 4 until a uniform emulsion was formed. The emulsion was then sonicated for 5 minutes at room temperature (Grant Ultrasonic Bath XB2).

The resultant emulsion was divided into 0.5 ml volumes and centrifuged at 14,000 RPM for 2.5 hours, after which the supernatant was removed and remaining liposome pellet washed with deionised water. This procedure was repeated three times to remove buffer.

0.5 ml of deionised water, pH adjusted to 8.5 with NaOH(aq), was added to 100 units of BT (Botox® Allergan Inc, USA) and the solution left to stand at room temperature for 20 minutes.

BT solution was then added to a vial containing the liposome pellet, slowly shaken on an orbital shaker (IKA KS130 Basic Orbital Shaker) for 2.5 hours, 160 cycles/minute.

Polysorbate 20 was then added (equivalent 7% w/w), shaking conditions were maintained throughout and continued for a further 30 minutes after addition.

Once the composition was prepared it was visually examined to determine whether the surfactant component had solubilised the emulsion in the aqueous media. The clarity of the mixture was categorised as being clear if there was no significant visible opacity to the naked eye.

The viscosity of the solution was then modified by the addition of 1.5% Natrasol® 250 HBXR, a slow shaking was maintained at room temperature for a further 30 minutes. Sample was then transferred to cold storage ˜4° C., for 24 hours, prior to use.

0.45 ml of the BT containing gel (approximately 90 units of Botox®) was applied to the right hand forehead of Subject 2 and 0.05 ml of the BT containing gel (approximately 10 units of Botox®) was applied to the left hand upper lip area of Subject 2 (right side of photographs in FIG. 6c). Both test areas were immediately occluded under PVC film for 30 minutes.

### Surfactant

Protasorb L-20 (Polysorbate-20) was as described in Example 2.

### Lipid

Vav S-70 was used as described in Formulation 7.1.

### Viscosity Modifier

Natrasol® 250 HBXR (Hydroxyethylcellulose) supplied by Ashland Aqualon Functional Ingredients (USA) CAS: 9004-62-0.

### Botulinum Neurotoxin Type A

BOTOX® (Botulinum Neurotoxin Type A) supplied by Allergan Inc., USA.

Results from Formulations 7.1 and 7.2

4 Weeks after topical application of BT containing gels 7.1 and 7.2 both subjects were reassessed for hydrosis at the site of application. A reduction in staining was visible—indicating a reduction in hydrosis is visible in both subjects in areas treated with BT (see FIGS. 6b and 6c).

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

Unless specifically stated otherwise, all ratios and proportions are given on a weight to weight basis.

1. A formulation comprising BT, lipid and surfactant, characterised in that the lipid and surfactant are in the form of macromolecular assemblies of less than 100 nm in diameter.

2. The formulation according to claim 1 characterised in that the surfactant has an HLB number of less than 20

3. The formulation according to claim 2 characterised in that the surfactant has an HLB number in the range of about 10.5 to about 17.5.

4. The formulation according to claim 1 characterised in that the surfactant is an ether surfactant.

5. The formulation according to claim 1 characterised in that the surfactant is an ester surfactant.

6. The formulation according to claim 1 characterised in that the surfactant is an ionic surfactant.

7. The formulation according to claim 1 characterised in that the surfactant is a copolymer of styrene and maleic acid.

8. The formulation according to claim 1 in which the ratio of surfactant to lipid is at least 0.5:1 on a weight basis.

9. The formulation according to claim 1 in which the ratio of surfactant to lipid is 25:1 or lower on a weight basis.

10. The formulation according to claim 1, wherein the macromolecular assemblies are less than 75 nm in diameter.

11. The formulation according to claim 1 presented as a unit dose containing 5-500 U of BT.

12. The formulation according to claim 1 wherein the BT is a natural BT polypeptide.

13. The formulation according to claim 1 wherein the BT is polypeptide having at least 90% identity to a sequence selected from SEQ ID Nos: 1-11.

14. A method for the manufacture of a formulation according to claim 1, comprising the steps of:
(i) Preparing an aqueous emulsion of lipid and BT; and
(ii) Mixing surfactant with the aqueous lipid/BT emulsion;
such that macromolecular assemblies are formed.

16. The method according to claim 14, wherein the surfactant is in aqueous solution.

17. The method according to claim 14, wherein the aqueous emulsion of lipid and BT is prepared by:
(a) creating of an emulsion of acidic liposomes with internal aqueous phases of pH 3-5
(b) extracting the acidic liposomes from the external buffer solution
(c) if necessary, disassociate carrier protein haemagglutinin from BT by adjustment of a solution of BT to pH to 7.5-9
(d) add disassociated BT solution at pH 7.5-9 to acidic liposomes.

18. A method for the manufacture of a formulation according to claim 1 comprising the step of mixing macromolecular assemblies of lipid and surfactant, characterised in that macromolecular assemblies are less than 100 nm in diameter with BT.

19. A kit of parts for the preparation of a formulation according to claim 1, comprising a lipid, a surfactant and BT.
